SEQUENCE LISTING (18 sequences)

SEQ ID NO: 1 - DNA, 136 bp, synthetic construct
  source 1..136: mol_type = other DNA
acggtcgtcaaatctgtcgtggtaggatctgtcgtggtaggatctgtcgtggtaactttggggttggataaatctacagtcttaagaactggctacgacggactgatcggatcgaataacgagtacacggtggtta

SEQ ID NO: 2 - DNA, 36 bp, synthetic construct
  source 1..36: mol_type = other DNA
taaccaccgtgtactcgttattcgatccgatcagtc

SEQ ID NO: 3 - DNA, 51 bp, synthetic construct
  source 1..51: mol_type = other DNA
taaccaccgtgtactcgttattcgatccgatcagtccgtcgtagccagttc

SEQ ID NO: 4 - DNA, 66 bp, synthetic construct
  source 1..66: mol_type = other DNA
taaccaccgtgtactcgttattcgatccgatcagtccgtcgtagccagttcttaagactgtagatt

SEQ ID NO: 5 - DNA, 139 bp, synthetic construct
  source 1..139: mol_type = other DNA
acggtcgtcaaatctgtcgtggtaggatctgtcgtggtaggatctgtcgtggtaactttggggttggataaatctacagtcttaagaactggctacgacggactgataaacggatcgaataacgagtacacggtggtta

SEQ ID NO: 6 - DNA, 141 bp, synthetic construct
  source 1..141: mol_type = other DNA
acggtcgtcaaatctgtcgtggtaggatctgtcgtggtaggatctgtcgtggtaactttggggttggataaatctacagtcttaagaactggctacgacggactgataaaaacggatcgaataacgagtacacggtggtta

SEQ ID NO: 7 - DNA, 143 bp, synthetic construct
  source 1..143: mol_type = other DNA
acggtcgtcaaatctgtcgtggtaggatctgtcgtggtaggatctgtcgtggtaactttggggttggataaatctacagtcttaagaactggctacgacggactgataaaaaaacggatcgaataacgagtacacggtggtta

SEQ ID NO: 8 - DNA, 66 bp, synthetic construct
  source 1..66: mol_type = other DNA
  modified_base 22: mod_base = OTHER; note = 2-(methoxymethyl)tetrahydrofuran-3-yl methyl hydrogen phosphate
taaccaccgtgtactcgttatncgatccgatcagtccgtcgtagccagttcttaagactgtagatt

SEQ ID NO: 9 - DNA, 136 bp, synthetic construct
  source 1..136: mol_type = other DNA
acggtcgtcaaatctgtcgtggtaggatctgtcgtggtaggatctgtcgtggtaactttggggttggataaatctacagtcttaagaactggctttttttgactgatcggatcgaataacgagtacacggtggtta

SEQ ID NO: 10 - DNA, 66 bp, synthetic construct
  source 1..66: mol_type = other DNA
taaccaccgtgtactcgttattcgatccgatcagtcaaaaaaagccagttcttaagactgtagatt

SEQ ID NO: 11 - RNA, 100 nt, synthetic construct
  source 1..100: mol_type = other RNA
tcgccaacagctccgcgcgagcacgagcgaagtcaactgctggacacctgacgacctgatccgtgattgcagggtttgcactagctgcgccgaacatgcg

SEQ ID NO: 12 - RNA, 40 nt, synthetic construct
  source 1..40: mol_type = other RNA
cgcatgttcggcgcagctagtgcaaaccctgcaatcacgg

SEQ ID NO: 13 - RNA, 60 nt, synthetic construct
  source 1..60: mol_type = other RNA
cgcatgttcggcgcagctagtgcaaaccctgcaatcacggatcaggtcgtcaggtgtcca

SEQ ID NO: 14 - RNA, 80 nt, synthetic construct
  source 1..80: mol_type = other RNA
cgcatgttcggcgcagctagtgcaaaccctgcaatcacggatcaggtcgtcaggtgtccagcagttgacttcgctcgtgc

SEQ ID NO: 15 - DNA, 76 bp, synthetic construct
  source 1..76: mol_type = other DNA
ggggttggataaatctacagtcttacgcgctggctacgacggactgatcggatcgaataacgagtacacggtggtt

SEQ ID NO: 16 - DNA, 51 bp, synthetic construct
  source 1..51: mol_type = other DNA
aaccaccgtgtactcgttattcgatccgatcagtccgtcgtagccagcgcg

SEQ ID NO: 17 - DNA, 51 bp, synthetic construct
  source 1..51: mol_type = other DNA
aaccaccgtgtactcgttattcgatccgatcagtccgtcgaagccagcgcg

SEQ ID NO: 18 - DNA, 51 bp, synthetic construct
  source 1..51: mol_type = other DNA
aaccaccgtgtactcgttattcgatccgatcagtccgtcagagccagcgcg

(19) United States
(12) Patent Application Publication
(10) Pub. No.: US 2024/0410829 A1
(43) Pub. Date: Dec. 12, 2024

(54) GRAPHENE ENERGY TRANSFER WITH VERTICAL NUCLEIC ACIDS

(71) Applicant: Ludwig-Maximilians-Universität München, München (DE)

(72) Inventors: Philip TINNEFELD, München (DE); Alan M. SZALAI, München (DE); Lars RICHTER, München (DE); Izabela KAMINSKA, München (DE); Giovanni FERRARI, München (DE)

(21) Appl. No.: 18/672,616

(22) Filed: May 23, 2024

Related U.S. Application Data
(60) Provisional application No. 63/503,747, filed on May 23, 2023.

(51) Int. Cl. G01N 21/64; C12Q 1/6834

(52) U.S. Cl. CPC G01N 21/6428; C12Q 1/6834; G01N 21/6402; G01N 21/6458; G01N 2021/6432

(57) ABSTRACT

A construct comprising a graphene substrate and a hybrid molecule comprising a single-stranded nucleic acid segment and a double-stranded nucleic acid segment, in which the hybrid molecule is immobilized to the graphene substrate by the single stranded nucleic acid segment, and in which the double-stranded nucleic acid segment comprises a linear segment comprising at least one base pair in perpendicular orientation to the graphene substrate at the junction where the double-stranded nucleic acid segment and the single-stranded nucleic acid segment join. In addition, a method for producing such a construct, the construct as produced by the production method of the invention, a method for measuring quenching efficiency by means of measuring the fluorescence lifetime or fluorescence intensity in such a construct, as well as a microscopy system for measuring relative fluorescence lifetime in such a construct.

### CROSS-REFERENCE TO RELATED APPLICATIONS

This application claims the benefit of U.S. Provisional Application No. 63/503,747, filed on May 23, 2023, which is herein incorporated by reference in its entirety.

### SEQUENCE LISTING

The instant application contains a Sequence Listing which has been submitted electronically in XML format and is hereby incorporated by reference in its entirety. Said XML copy, created on Aug. 6, 2024, is named VOS_006_US1-Sequence_Listing and is 18,743 bytes in size.

### BACKGROUND OF THE INVENTION

The present invention relates to a construct comprising a graphene substrate and a hybrid molecule comprising a single-stranded nucleic acid segment and a double-stranded nucleic acid segment, wherein the hybrid molecule is immobilized to the graphene substrate by the single-stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join. The present invention further relates to a method for producing such a construct, the construct as produced by the production method of the invention, a method for measuring quenching efficiency by means of measuring the fluorescence lifetime and/or fluorescence intensity in such a construct, as well as a microscopy system for measuring relative fluorescence lifetime in such a construct.

Visualizing the structures of molecules and their dynamic conformational changes is central for understanding the molecular foundations of life. Over the past two decades, single-molecule (sm) Fluorescence Resonance Energy Transfer (FRET) has become a workhorse of dynamic structural biology as it provides conformational states and dynamics of biomolecules directly and without synchronization necessary for ensemble methods (Lerner E, et al., Science, 2018, 359:1-12). Many exciting molecular mechanisms especially on nucleic acids and nucleic acid-protein interactions could be revealed such as DNA scrunching in transcription initiation or regulation in DNA metabolic processes (Kapanidis A N, et al., Science, 2006, 314:1144-1147; Zhou R, et al., Nature Chemical Biology, 2019, 15: 269-275).

Graphene and DNA have been combined in various applications and devices. However, different aspects of the interactions between these components are still unknown. An exemplary case is the orientation of double-stranded DNA (dsDNA) on graphene. While it is known that single-stranded DNA (ssDNA) adsorbs on graphene via non-covalent TT-stacking interactions, this does not happen with dsDNA because the bases are compromised in base pairing forming the double helix. There are few theoretical studies that predicted that dsDNA could stand perpendicular to graphene (Gu Z, et al., ACS Nano, 2017, 11:3198-3206; Zhao X. The Journal of Physical Chemistry C, 2011, 115:6181-6189; Li B, et al., The Journal of Physical Chemistry C, 2020, 124:3332-3340). Specifically, two possible weak binding conformations were suggested, one that is lying on graphene and one in which the double strand end-stacks on graphene. However, both conformations are not considered stable interactions with graphene in room temperature solution conditions as revealed by fluorescence assays. dsDNA is not stably attaching to graphene and there have not been theoretical studies of single-stranded/double stranded junctions.

Many sensors were developed in the last decade based on the differential interaction between ssDNA and dsDNA with graphene or graphene oxide (GO) (Kaminska I, et al., Nano Letters, 2019, 19:4257-4262; Ghosh A, et al., Nature Photonics, 2019, 13:860-865). In these approaches, ssDNA modified with a fluorophore is first immobilized on graphene or GO, resulting in a nearly 100% quenching of the fluorescence of a dye molecule. When the target molecule, i.e., the complementary strand, is present, it hybridizes with the dye-modified ssDNA. The formed dsDNA desorbs from the graphene or GO surface and hence the fluorescence signal is restored. Most of these works use GO instead of graphene because it behaves similarly in terms of fluorescence quenching, and the material is cheaper. Although GO has also other advantages such us water solubility and rich chemistry due to the presence of oxygen containing functional groups which can be further used for the incorporation of various moieties, there are no systematic studies on the distance dependency of the fluorescence quenching (energy transfer efficiency) of fluorophores in the presence of GO, which is mainly caused by the flaky nature of GO, i.e., the small surface area and inability to easily determine the number of layers. Lacking an appropriate calibration curve, it is not possible to calculate the distance between a dye molecule and GO based on the fluorescence lifetime/intensity measurements. Yet, these works neither determined whether a hybrid ssDNA-dsDNA construct would be vertically oriented when added to graphene, nor explored the advantages that such orientation can offer for new devices and applications.

Only one work used a ssDNA-dsDNA hybrid molecule to immobilize DNA on the surface, but the authors used GO instead of graphene and did not calculate or study the exact orientation of the dsDNA segment (Piao Y, et al., Chemical Communications, 2011, 47:12149-12151). In this document, the DNA is showing away from graphene due to negative charge repulsion of the negatively charged DNA and the negatively charged graphene oxide. This would however not lead to a vertical orientation. The distance dependence shown is also not supporting a vertical orientation on GO. A second relevant paper also includes hybrid ssDNA-dsDNA molecules on GO, but the authors do not discuss the orientation of the double helix and focused their research on how the ssDNA segment affected the affinity of the whole molecule to the surface (Park J S, et al., Langmuir, 2014, 30:12587-12595).

### SUMMARY OF THE INVENTION

Graphene and DNA have been combined in various applications and devices, however, different aspects of the interactions between these components are still unknown. An exemplary case is the orientation of double-stranded DNA on graphene. The present inventors have surprisingly and unexpectedly found that the double-stranded part of a single-stranded/double-stranded nucleic acid hybrid molecule stands vertically on graphene. As evident from the appended Examples, the lowest double-stranded nucleotide is firmly attached to the graphene in order for double-stranded DNA or double-stranded RNA to adopt a vertical orientation when immobilized on a graphene substrate by a single-stranded toehold. Accordingly, provided are improvements based on the identification of the fixed vertical orientation of a double-stranded nucleic acid segment of a hybrid molecule on graphene, which offers various potential applications, especially in the exploration of structural and functional features of nucleic acid sequences.

The present invention provides a construct comprising a graphene substrate and a hybrid molecule. The hybrid molecule according to the present invention comprises a single-stranded nucleic acid segment and a double-stranded nucleic acid segment. As such, the hybrid molecule comprises a segment consisting of a single strand of a nucleic acid sequence and a segment consisting of a double strand of a nucleic acid sequence, wherein the single strand of the nucleic acid sequence forms one strand of the double strand nucleic acid sequence. Herein, the term nucleic acid is used as commonly understood in the art and is further explained in detail in the ongoing description.

In the present invention, the hybrid molecule of the construct of the invention is immobilized to the graphene substrate through the single-stranded nucleic acid segment. Herein, the term “immobilized” is used as commonly understood in the art and is further explained in detail in the ongoing description. The length of the single-stranded nucleic acid segment is not particularly limited; however, the single-stranded nucleic acid should be at least 5 nucleotides in length to ensure sufficient immobilization onto the graphene substrate. In a preferred embodiment, the single-stranded nucleic acid segment is between 5 and 500, more preferably between 5 and 250, even more preferably between 5 and 100, and most preferably between 40 and 100 nucleotides in length.

Forming part of the hybrid molecule disclosed herein, the double-stranded nucleic acid segment comprises a linear segment comprising at least one base pair in perpendicular orientation to the graphene substrate at the junction where the double-stranded nucleic acid segment and the single-stranded nucleic acid segment join. In the present invention, the at least one base pair in perpendicular orientation as comprised in the double-stranded nucleic acid segment is considered being the first base pair near the junction of the double-stranded nucleic acid segment and the single-stranded nucleic acid segment. The number of base pairs in the linear segment is not particularly limited. Exemplary, the linear segment may comprise, including the at least one base pair which is in perpendicular orientation to the graphene substrate, two, three, four, or more base pairs. In one embodiment, the linear segment comprising at least one base pair in perpendicular orientation to the graphene substrate comprised in the double-stranded nucleic acid segment is at least 15 base pairs, preferably at least 20 base pairs, most preferably at least 36 base pairs long.

In a preferred embodiment of the present invention, the first base pair of the double-stranded nucleic acid segment of the hybrid molecule at the junction, i.e., the at least one base pair in perpendicular orientation to the graphene substrate, is immobilized to the graphene substrate. That is, the first base pair is considered to naturally attach to the graphene substrate.

In the present invention, the graphene substrate as comprised in the construct of the invention refers to a substrate comprising graphene as 2D hexagonal carbon lattice. In the context of the present invention, graphene comprised in the graphene substrate can be provided as graphene monolayer or multiple graphene layers such as bi- or tri-layers. The number of graphene layers is not particularly limited, provided that the graphene layer(s) as comprised in the graphene substrate is/are suitable to be comprised in the construct of the invention. In a preferred embodiment, the at least one graphene layer is located at/provides the upper part of the graphene substrate, i.e., is in contact with the hybrid molecule to be reacted with.

In one embodiment, the graphene substrate comprises at least a monolayer of graphene on a matrix. That is, the graphene substrate may further comprise a matrix different from graphene in addition to the graphene. The skilled person appreciates that the number of graphene layers in this embodiment is not particularly limited. In the present invention, the matrix is considered to provide the base of the graphene substrate such that graphene is immobilized on top of the matrix. The invention does not exclude further components as part of the graphene substrate, e.g., below the matrix or as a base for the matrix. The matrix is not particularly limited and may be made from one material or a mixture of more than one material (e.g., two, three, four or more materials) which are suitable to serve as a matrix in the graphene substrate according to the invention. In the present invention, it is preferred that the matrix is made from a transparent (i.e., pervious to light) material, even more preferably from glass. Most preferably, the glass is borosilicate glass. In this embodiment, the thickness of the glass is not particularly limited. Preferentially, the glass may have a in thickness of about 170 μm, which is a typical thickness of thin cover glass. In a particularly preferred embodiment, the graphene substrate consists of a monolayer of graphene on glass as the matrix.

In one embodiment of the invention, the nucleic acid of either one or both of the single-stranded nucleic acid segment and the double-stranded nucleic acid segment is selected from the group consisting of DNA or RNA. Herein, the terms “DNA” and “RNA” are used as commonly understood in the art and are further explained in detail in the ongoing description. In a preferred embodiment, the single-stranded nucleic acid segment is a single-stranded DNA segment, and the double-stranded nucleic acid segment is a double-stranded DNA segment.

In a further embodiment of the invention, the double-stranded nucleic acid segment of the hybrid molecule as described herein comprises at least one bend and a further linear segment extending from the bend having a non-perpendicular orientation to the graphene substrate. The number of bends as comprised in the double-stranded nucleic acid segment is not particularly limited, e.g., the double-stranded nucleic acid segment of the invention may comprise one or more, e.g., two, three, four or more bends. A bend as described herein is considered to force the nucleic acid sequence into an angle relative to the graphene substrate which is divergent from the 90° angle of the above-described perpendicular linear segment as comprised in the double-stranded nucleic acid segment of the invention to the graphene substrate. In the present invention, it is considered that the at least one bend as optionally comprised in the double-stranded nucleic acid segment is followed by a linear nucleic acid segment which extends from the bend and has a non-perpendicular orientation to the graphene substrate following the bending angle. In a certain embodiment, the double-stranded nucleic acid segment of the hybrid molecule as described herein comprises one or more bends and a linear segment extending from each of the one or more bends.

As bending of nucleic acids may occur in response to several events, the skilled person is aware that the cause of bending is not particularly limited in the present invention. In a preferred embodiment, the bend originates from the presence of unpaired mismatches, abasic (AP) sites, A-tracts, nicks, or binding of at least one protein that interact with nucleic acid. More preferably, the at least one bend originates from unpaired adenine bulges or nucleic acid-interacting proteins. Of the nucleic acid-interacting proteins, mutant Escherichia coli endonuclease IV is particularly preferred.

In one embodiment of the invention, the double-stranded nucleic acid segment comprised in the construct of the invention comprises at least one photoluminescent particle. The photoluminescent particle is not particularly limited, as long as it is subject to photoluminescence. In a preferred embodiment, the at least one photoluminescent particle is selected from the group consisting of quantum dots, fluorescent beads and fluorescence dyes (fluorophores). More preferably, the photoluminescent particle is a quantum dot or a fluorophore, most preferably a fluorophore. Specific types of photoluminescent particles, e.g., specific types of quantum dots or fluorophores are well-known in the art and the invention is not particularly limited in this regard. The skilled person appreciates that the photoluminescent particles may emit light in different spectral regions, e.g., green emission and red emission. Exemplary photoluminescent particles are listed in the ongoing description.

In the present invention, the at least one photoluminescent particle is understood as to refer to the double-stranded nucleic acid segment comprising one, two, three, four or more photoluminescent particles. The total number of photoluminescent particles as comprised by the double-stranded nucleic acid segment is not particularly limited.

In one embodiment, the at least one photoluminescent particle as comprised by the double-stranded nucleic acid segment may be directly attached to the double-stranded nucleic acid segment. In another embodiment, the at least one photoluminescent particle as comprised by the double-stranded nucleic acid segment may be indirectly attached to the double-stranded nucleic acid segment. If more than one photoluminescent particle is attached to the double-stranded nucleic acid segment, the skilled person understands that the invention covers the embodiment of mixed attachment, i.e., directly and indirectly attached photoluminescent particles.

The terms “directly” and “indirectly” are used as commonly understood in the art and are further explained in detail in the ongoing description. In the present invention, indirect attachment is considered to refer to binding of the at least one photoluminescent particle to a protein that is itself bound to the double-stranded nucleic acid segment. Such DNA-binding proteins are well-known in art and the present invention is not particularly limited to a specific type. However, a preferred DNA-binding protein is 06-alkylguanine DNA alkyltransferase (AGT) which has been exemplary tested in the examples.

The at least one photoluminescent particle as comprised by the double-stranded nucleic acid segment, which may be directly or indirectly attached to the double-stranded nucleic acid segment, may be attached to any base of the nucleic acid. Accordingly, the position of attachment to the nucleic acid is not particularly limited.

In the specific embodiment, in which the at least one photoluminescent particle as comprised by the double-stranded nucleic acid segment is directly attached to the double-stranded nucleic acid segment, it is preferred that the at least one photoluminescent particle is directly attached specifically to the last base pair of the double-strand DNA segment extending from the junction of the single-stranded nucleic acid segment and the double-stranded nucleic acid segment.

In one embodiment of the invention, the construct of the invention is characterized in that the distance of the at least one photoluminescent particle as described herein to the graphene substrate is at least 5 nm. The skilled person understands that the at least 5 nm refer to the smallest possible distance between any photoluminescent particle as comprised by the double-stranded nucleic acid segment and the surface of the graphene substrate.

The present invention also relates to a method for producing the construct of the invention. Said method comprises the step of (i) or (ii): (i) mixing a graphene substrate and a hybrid molecule comprising a single-stranded nucleic acid segment and a double-stranded nucleic acid segment to allow immobilization of the single-stranded nucleic acid segment to the graphene substrate; or (ii) adding a first single-stranded nucleic acid to a graphene substrate to allow attachment of the first single-stranded nucleic acid to the graphene substrate, and adding a second single-stranded nucleic acid complementary to the first single-stranded nucleic acid to form a hybrid molecule comprising a single-stranded nucleic acid segment and a double-stranded nucleic acid segment. In the context of (ii) it should be noted that a part of the single-stranded nucleic acid segment stays attached to the graphene substrate. That is, only the complementary part of the single-stranded nucleic acid sequence detaches from the graphene substrate when the hybrid molecule is formed by the complementary part of the first single-stranded nucleic acid and the second single-stranded nucleic acid.

In the present invention, it is preferred that the method for producing the construct of the invention comprises the step (i) as described above. The skilled person will appreciate that the definitions and embodiments as referred to in the context of the construct of the invention are applicable to the method of the invention for producing the construct.

In a preferred embodiment, the construct as produced by/obtainable by the above disclosed production method of the invention is characterized by having a double-stranded nucleic acid segment comprising a linear segment comprising at least one base pair in perpendicular orientation to the graphene substrate at the junction where the double-stranded nucleic acid segment and the single-stranded nucleic acid segment join. The skilled person appreciates that the definitions as provided for the construct of the invention are applicable to the construct as produced by the method of the invention.

The method for producing the construct of the invention as disclosed herein may optionally comprise a step of (i) attaching at least one photoluminescent particle to the single-stranded nucleic acid, specifically to the part thereof that forms the double-stranded nucleic segment, or (ii) attaching at least one photoluminescent particle to the double-stranded DNA segment. The time point of attaching the at least one photoluminescent particle is not particularly limited, provided that the construct as produced by the production method of the invention comprises at least one photoluminescent particle in the double-stranded nucleic acid segment as described herein. In a preferred embodiment, the method comprises the step of mixing a graphene substrate as disclosed herein and a hybrid molecule as disclosed herein, wherein the double-stranded nucleic acid segment of the hybrid molecule is labelled with at least one photoluminescent particle before mixing with the graphene substrate.

The present invention encompasses the construct comprising a graphene substrate and a hybrid molecule comprising a single-stranded nucleic acid segment and a double-stranded nucleic acid segment as obtainable by the method for producing the construct as disclosed herein.

The present invention further relates to the use of the construct as described herein (i.e., the construct of the invention and the construct as obtainable by the method of the invention). The skilled person will appreciate that the invention offers various potential applications. In a preferred embodiment, the invention relates to the use of the construct as described herein for measuring fluorescence lifetime and/or fluorescence intensity. Herein, the terms “fluorescence lifetime” or “fluorescence intensity” are used as commonly understood in the art and are further explained in detail in the ongoing description. A skilled person appreciates that the use of the construct as described herein for measuring fluorescence lifetime or fluorescence intensity extends to any secondary use that is associated with the use for measuring fluorescence lifetime and/or fluorescence intensity.

The invention further relates to a method for measuring quenching efficiency. In the present invention, the method for measuring quenching efficiency comprises the step of measuring quenching efficiency by means of measuring the fluorescence lifetime or fluorescence intensity in a construct of the invention or in a construct as obtainable by the production method of the invention. Means for measuring quenching efficiency by means of measuring the fluorescence lifetime or fluorescence intensity are known to a skilled person and are not particularly limited. In a preferred embodiment, quenching efficiency by means of measuring the fluorescence lifetime or fluorescence intensity is measured by using Fluorescence Lifetime Imaging Microscopy. In a non-limiting example, fluorescence lifetime can be determined by using the microscopy system of the invention.

The invention, thus, further relates to a microscopy system for measuring fluorescence lifetime. The microscopy system of the invention is a microscopy system for measuring fluorescence lifetime in a construct as described herein, wherein the double-stranded nucleic acid as comprised in the hybrid molecule comprises a photoluminescent particle as described herein. As such, the construct comprises a graphene substrate and a hybrid molecule comprising a single-stranded nucleic acid segment and a double-stranded nucleic acid segment, wherein the hybrid molecule is immobilized to the graphene substrate by the single stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join.

Particularly, the microscopy system may comprise: an excitation path, an objective, a detection path, a detector, and a Time Correlated Single Photon Counting unit; wherein the excitation path comprises a laser and is configured to produce a pulsed laser beam and direct the pulsed laser beam into the objective using one or more beam redirecting elements, the pulsed laser beam having a pulse width below 750 ps, preferably below 500 ps, more preferably below 200 ps; wherein the objective is configured to transmit the pulsed laser beam into a sample comprising the construct and to collect light from the sample, particularly light emitted from the construct; wherein the detection path is configured to direct light from the objective, particularly the light from the sample, to the detector using one or more beam redirecting elements; wherein the detector is configured to detect the light received from the detection path, particularly the light from the sample that is directed onto, e.g., focused on, the detector using a lens; and wherein the microscopy system is configured to measure an arrival time of individual photons at the detector after a laser pulse, using the Time Correlated Single Photon Counting unit.

The excitation path may be configured to shape the laser beam exiting the laser according to measurement requirements. A pulsed laser beam as used herein may comprise and/or consist of any number of pulses. For example, a laser beam may consist of a single pulse, two pulses, three pulses, or a higher number of pulses. As known in the art, when travelling along the detection path and/or the objective, the laser beam may be subject to intentional changes and/or unintentional changes such as intensity losses, etc. The objective may comprise one or more lenses. Typically, the objective comprises several lenses. The beam redirecting elements may be of any kind known in the art. Particularly, the beam redirecting elements may include one or more mirrors such as metallic mirrors and dichroic mirrors, one or more acousto-optical elements, one or more lenses, and combinations thereof.

The excitation path and/or the detection path may comprise further optical elements, e.g., one or more irises or apertures, one or more filters, one or more beam combiners, e.g., dichroic mirrors, one or more beam splitters, e.g., dichroic mirrors, means for adjusting the average power in the laser beam, one or more shutters for blocking light travelling along the excitation path/detection path, and combinations thereof. For example, in some embodiments, the average power of the pulsed laser beam is 10 μW or less, preferably 6 μW or less, more preferably 4 μW or less at the position of the objective. The microscopy system may be configured to set the average power according to the requirements of an application.

In some embodiments, the microscopy system may be set to deliver a laser beam with an average power at the location of the objective that results in non-saturating conditions for the excitation of the fluorophores used in the application. For example, 4 μW may be used with various fluorescent dyes in the UV, visible and IR spectral regions including fluorescent dyes from the classes of coumarines, rhodamines, cyanines, xanthenes, pyrenes, oxazines and pyronins.

The excitation path may include any suitable means for shaping the beam, for example one or more mirrors such as metallic mirrors and dichroic mirrors, one or more acousto-optical elements, one or more lenses, one or more irises or apertures, and combinations thereof. One or more elements that are used for beam shaping may also be used for beam redirection and vice versa. The detection path may be configured to focus the light from the objective into a point-like detector, preferably an avalanche photodiode detector. Alternatively, or additionally, the detection path may be configured to direct the light from the objective onto a camera configured for fluorescence lifetime imaging.

The microscopy system may be configured to control the polarisation of the laser beam and/or the light in the detection path. For example, the microscopy system may comprise one or more waveplates, for example quarter and/or half wave plate(s) for that purpose. The microscopy system may be configured to provide laser beams of different central wavelengths to the sample. For example, the microscopy system may comprise a first laser configured to emit a laser beam of a first central wavelength, and a second laser configured to emit a laser beam of a second central wavelength that differs from the first central wavelength. Everything described herein with reference to the excitation path also applies to excitation paths with two or more laser lines. The components of the microscopy system may be configured to transmit and reflect the wavelengths as appropriate. The microscopy system may be configured to combine the laser lines at one or more points in the excitation path. For example, the first and second laser beams may be combined at a certain point in the excitation path, e.g., via a dichroic mirror. For each laser beam that is combined with another beam one dichroic mirror may be used. Alternatively, or additionally, one or more of the lasers may be tuneable, i.e., be configured to emit laser beams of different central wavelengths. The detection path may be configured according to the laser lines of the excitation path. Particularly, the detection path may be configured to direct light with wavelengths according to fluorescence molecule emission spectra corresponding to the excitation wavelengths of the microscopy system to the detector(s).

As an alternative to the pulsed configuration of the microscopy system, a microscopy system configured for fluorescence lifetime determination in the frequency domain is envisaged.

The microscopy system may comprise any suitable number of excitation lines (i.e., excitation wavelengths and corresponding excitation paths) and corresponding spectral windows in the detection path according to the multiplexing needs of the application.

In particular, the invention relates to the following items:
1. A construct comprising a graphene substrate and a hybrid molecule comprising a single-stranded nucleic acid segment and a double-stranded nucleic acid segment, wherein the hybrid molecule is immobilized to the graphene substrate by the single-stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join.
2. The construct of item 1, wherein the nucleic acid is selected from the group consisting of DNA and RNA.
3. The construct of item 1 or 2, wherein the single-stranded nucleic acid segment is a single-stranded DNA segment, and the double-stranded nucleic acid segment is a double-stranded DNA segment.
4. The construct of any one of items 1 to 3, wherein the first base pair of the double-stranded nucleic acid segment of the hybrid molecule at the junction is immobilized to the graphene substrate.
5. The construct of any one of items 1 to 4, wherein the graphene substrate comprises at least a monolayer of graphene on a matrix.
6. The construct of any one of items 1 to 5, wherein the double-stranded nucleic acid segment comprises at least one bend and a further linear segment extending from the bend having a non-perpendicular orientation to the graphene substrate.
7. The construct of item 6, wherein the bend originates from the presence of unpaired mismatches, abasic sites, A-tracts, nicks, or binding of different proteins that interact with nucleic acid, preferably from unpaired adenine bulges or binding of a mutant Escherichia coli endonuclease IV.
8. The construct of any one of items 1 to 7, wherein the double-stranded nucleic acid segment comprises at least one photoluminescent particle.
9. The construct of item 8, wherein the at least one photoluminescent particle is attached directly to the double-strand nucleic acid segment.
10. The construct of item 9, wherein the at least one photoluminescent particle is attached to the last base pair of the double-strand nucleic acid segment extending from the junction of the single-stranded nucleic acid segment and the double-stranded nucleic acid segment.
11. The construct of item 8, wherein the at least one photoluminescent particle is attached indirectly to the double-strand nucleic acid segment via a protein bound to the double-strand DNA segment.
12. The construct of any one of items 8 to 11, wherein the at least one photoluminescent particle is a fluorophore or a quantum dot, preferably a fluorophore.
13. The construct of any one of items 1 to 12, wherein the linear segment comprised in the double-stranded nucleic acid segment is at least 15 base pairs, preferably at least 20 base pairs, most preferably at least 36 base pairs long.
14. The construct of any one of items 1 to 13, wherein the single-stranded nucleic acid segment is between 5 and 100 nucleotides, preferably between 40 and 100 nucleotides in length.
15. The construct of any one of items 1 to 14, wherein the distance of the photoluminescent particle to graphene is at least 5 nm.
16. A method for producing the construct of any one of items 1 to 15, wherein the method comprises the step of (i) or (ii):
   (i) mixing a graphene substrate and a hybrid molecule comprising a single-stranded nucleic acid segment and a double-stranded nucleic acid segment to allow immobilization of the single-stranded nucleic acid segment to the graphene substrate; or
   (ii) adding a first single-stranded nucleic acid to a graphene substrate to allow attachment of the first single-stranded nucleic acid to the graphene substrate, and adding a second single-stranded nucleic acid complementary to the first single-stranded nucleic acid to form a hybrid molecule comprising a single-stranded nucleic acid segment and a double-stranded nucleic acid segment.
17. The method of item 16, wherein the obtained construct is characterized by having a double-stranded nucleic acid segment comprising a linear segment comprising at least one base pair in perpendicular orientation to the graphene substrate at the junction where the double-stranded nucleic acid segment and the single-stranded nucleic acid segment join.
18. A construct comprising a graphene substrate and a hybrid molecule comprising a single-stranded nucleic acid segment and a double-stranded nucleic acid segment as obtainable by the method of item 16 or item 17.
19. Use of the construct of any one of items 8 to 15 or the construct as obtainable by method item 16 or item 17 for measuring fluorescence lifetime or for measuring fluorescence intensity.
20. A method for measuring quenching efficiency by means of measuring the fluorescence lifetime and/or fluorescence intensity in a construct of any one of items 8 to 15 or as obtainable by method item 16 or item 17.
21. The method of item 20, wherein fluorescence lifetime and/or fluorescence intensity is measured by using Fluorescence Lifetime Imaging Microscopy.
22. A microscopy system for measuring fluorescence lifetime in a construct of any one of items 8 to 15 or 18, the construct comprising a graphene substrate and a hybrid molecule comprising a single-stranded nucleic acid segment and a double-stranded nucleic acid segment, wherein the hybrid molecule is immobilized to the graphene substrate by the single-stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join.
23. The microscopy system of item 22, comprising:
   an excitation path, an objective, a detection path, a detector, and a Time Correlated Single Photon Counting unit;
   wherein the excitation path comprises a laser and is configured to produce a pulsed laser beam and direct the pulsed laser beam into the objective using one or more beam redirecting elements, the pulsed laser beam having a pulse width below 750 ps, preferably below 500 ps, more preferably below 200 ps;
   wherein the objective is configured to transmit the pulsed laser beam into a sample comprising the construct and to collect light from the sample, particularly light emitted from the construct;
   wherein the detection path is configured to direct light from the objective, particularly the light from the sample, to the detector using one or more beam redirecting elements;
   wherein the detector is configured to detect the light received from the detection path, particularly the light from the sample, that is directed onto, e.g., focused on, the detector using a lens; and wherein the microscopy system is configured to measure an arrival time of individual photons at the detector after a laser pulse, using the Time Correlated Single Photon Counting unit.
24. The microscopy system of item 23, wherein the average power of the pulsed laser beam is 10 μW or less, preferably 6 μW or less, more preferably 4 μW or less at the position of the objective.
25. The microscopy system of item 23 or 24, wherein the detection path is configured to focus the light from the objective into a point-like detector, preferably an avalanche photodiode detector

### BRIEF DESCRIPTION OF DRAWINGS

FIG. 1. Description of the working principle and the theoretical basis. a) Sketch of the GETvNA immobilization strategy with a hybrid molecule consisting of a single-stranded DNA (ssDNA) segment bound to graphene and a double-stranded DNA (dsDNA) segment pointing perpendicular from graphene. At the end of the dsDNA segment, a fluorophore is attached to the hybrid molecule represented by the shining star. The right side visualizes the increasing fluorescence lifetime T with increasing distance z of the fluorophore to graphene. b) Theoretical dependence of the relative fluorescence intensity I/I₀ or similarly the relative fluorescence lifetime _{T}/_{T}_{₀} on the distance z, following a d⁻⁴ power law. The characteristic distance do when both relative values amount to 0.5 is given accordingly. c) Plots of the spatial precision σ_{CRB} in Ångström depending on the distance z for three different photon numbers N (lower plot). Dependence of the signal-to-background ratio (SBR) on the distance z.

FIG. 2. Benchmarking of the hybrid molecule. a) Sketch of the immobilization strategy for nucleic acids on graphene with the single-stranded segment, the double-stranded segment, and a Cy3B fluorophore as a shining star. The distance d of the fluorophore to graphene, the length L of the double-stranded segment and the angle α are denoted. b) Upper row and lower left panel: Fluorescence Lifetime Imaging Maps (FLIM) of three different hybrid molecule types with varying length L in base pairs (bp). Lower-right panel: Exemplary, normalized fluorescence decay histograms for the varying lengths depending on the time. c) Normalized histograms of the measured fluorescence lifetimes for the three lengths of the hybrid molecule types, as in FIG. 2b. For each histogram, a Gaussian fit is added. d) Normalized histograms of the calculated heights z for the three hybrid molecule types. The conversion of the measured fluorescence lifetime to the calculated heights is performed by employing formula (1). Gaussian plots are shown for the three histograms. e) Comparison of the experimental results and the theoretically expected lengths of dsDNA given by the Worm-like chain model as a function of the number of base pairs. f) Normalized histograms of the calculated angles for the three hybrid molecule types. The conversion of the calculated heights to the angles is realized by using formula (2). The inset depicts a hybrid molecule standing perpendicular to graphene. The same color code is applied in subfigures b), d) and f).

FIG. 3. GETvNA for angle measurements a) Sketch of the applied principle for measuring the bending of nucleic acids. The three versions of bulges are visualized at the height of the kink. b) Plot showing the calculated height z as a function of the angle of nucleic acid bending θ. c) Sketches of the hybrid molecule with varying sizes of the incorporated bulge. d) Histograms of the distances z for each size of the bulge. The histograms for the 3A and the 5A bulge are fitted by the sum of two Gaussians that are centered at different distances. The histogram for the 7A bulge contains a single Gaussian fit. e) Histograms of the calculated angle values corresponding to the different sizes of the bulge. Single or two Gaussian fits are included, as in FIG. 3d.

FIG. 4. GETvNA demonstrates the bending capability of endonucleases IV (Endo IV). a), b) Sketches of the recognition and bending of DNA by the enzyme Endo IV leading to a reduced distance of the fluorophore to graphene. The insets display the molecular structure of dsDNA segment with an abasic site (left) and Endo IV bending the DNA. c) Two intensity traces for unbent hybrid molecules. The respective angle is given in each plot on the lower-left side. d) Histogram of the calculated angles θ from all measurements. The two dashed lines visualize the center positions of a Gaussian fit for the unbent and bent angles, respectively. e) Intensity traces showing different dynamical behavior after Endo IV addition. f) Histogram of the calculated angles after Endo IV addition, with the two dashed lines representing the unbent and bent state as labelled.

FIG. 5. GETvNA showing the dynamics of AGT enzymes. a) Sketch of an AGT monomer moving along the dsDNA of a hybrid molecule. The inset visualizes the AGT monomer binding to dsDNA. b) Sketch of hybrid molecules with and without A-tract. c) Fluorescence trace of an AGT monomer moving along the dsDNA. The plot shows the derived distance z as a function of time. The region containing the A-tract is highlighted by the two dashed lines. d) Trace of the distance z for an AGT monomer diffusing along the dsDNA on a hybrid molecule without A-tract. Again, the region previously containing the A-tract is marked. e) Demonstration of the calculation of the enzyme/protein step size. f) Simplified diagram showing the enzyme moving in base pair steps along the dsDNA with time. g) Plot of the protein steps as a function of time. The dashed lines indicate single base pair steps. h) Histogram of the protein steps, with the dashed lines showing single Gaussian fits. The values including error on top provide the center position of each Gaussian fit. The thick solid line indicates the sum of the five fits.

FIG. 6. Graphene-Energy-Transfer with vertical nucleic acids applied to RNA. a) Fluorescence Lifetime Imaging Map (FLIM), b) fluorescence lifetime, and c) height distribution of single molecules above graphene attached to dsRNA of 80 bp (n=446). d) Fluorescence lifetime and e) height distribution of single-molecules above graphene attached to dsRNA of 40 bp (n=286). f) Fluorescence lifetime and g) height distribution of single-molecules above graphene attached to dsRNA of 60 bp (n=222).

FIG. 7. Detection of DNA mismatches. a) Height distribution of dsDNA having no DNA mismatch on graphene. b) Height distribution of the double-stranded DNA as in a) having one DNA mismatch. c) Height distribution of the double-stranded DNA as in a) having two DNA mismatches. d) Comparison of height distribution of the double-stranded DNA having one or two mismatches.

### DETAILED DESCRIPTION OF THE INVENTION

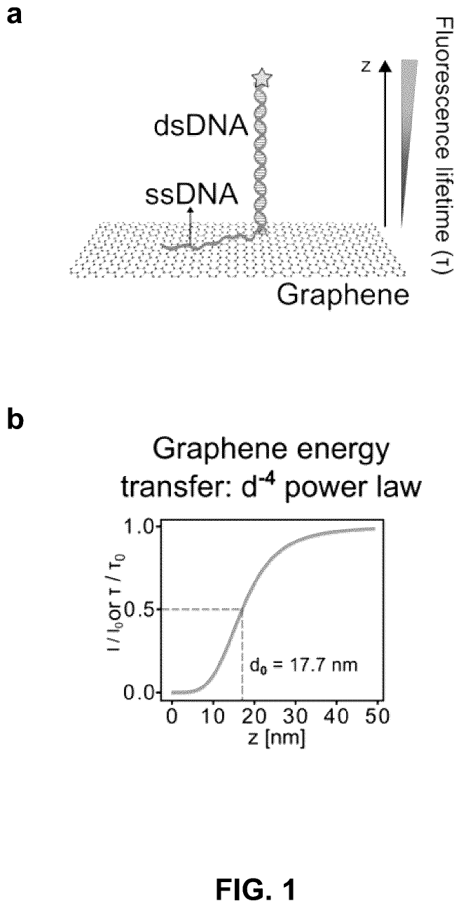

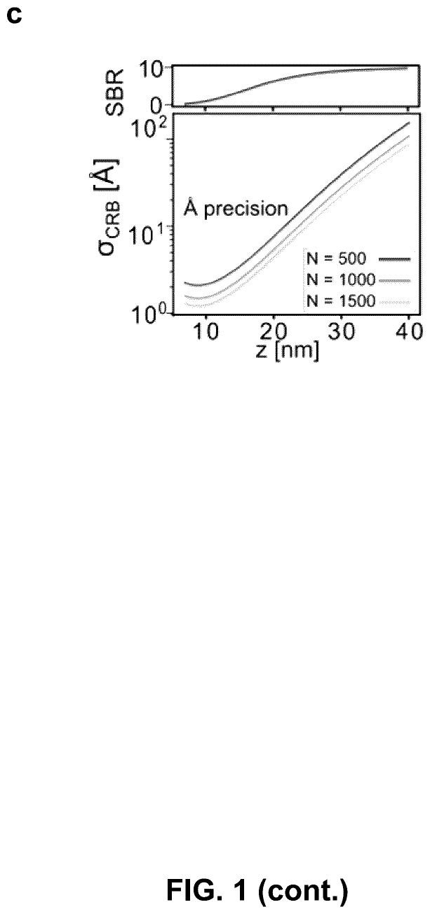

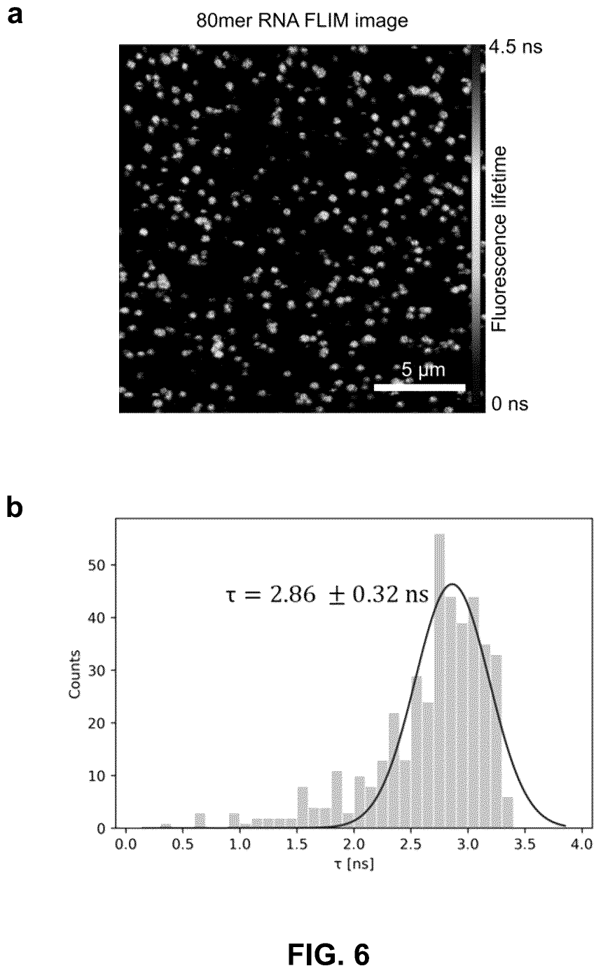

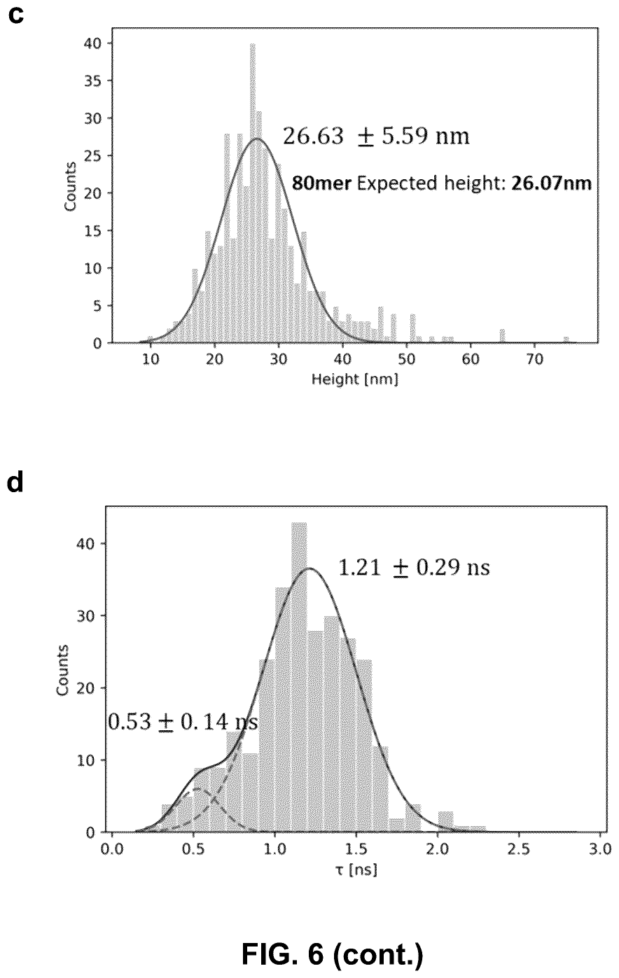

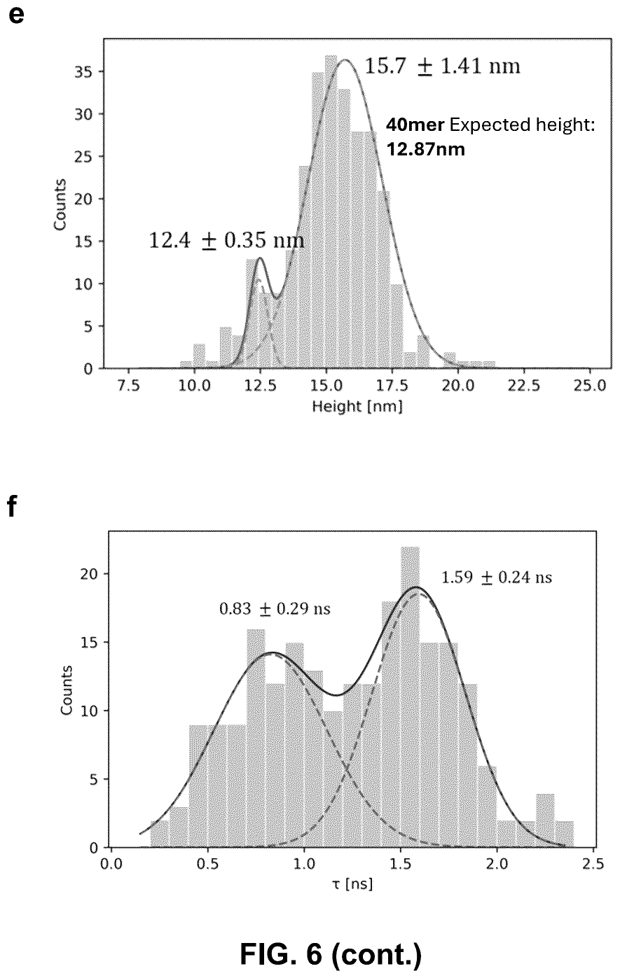

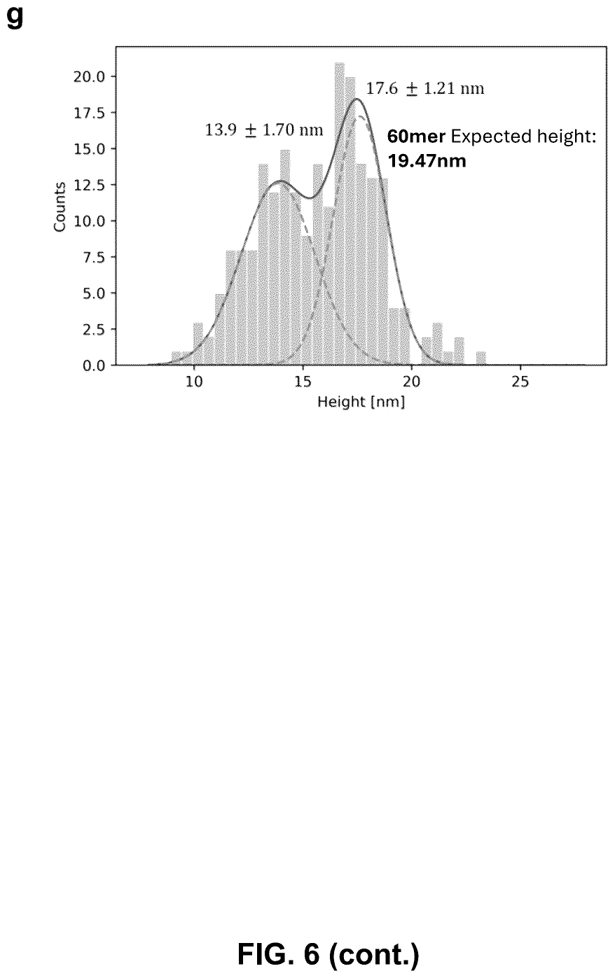

The present invention brings forth a new dynamic structural biology tool for nucleic acids and nucleic acid-protein interactions that is termed Graphene-Energy-Transfer with vertical Nucleic Acids (GETvNA). Single-molecule fluorescence experiments have been used to study nucleic acid orientation and it has been demonstrated that double-stranded DNA and RNA stands perpendicular to graphene. For DNA, this conformation is stable for weeks. The finding that the double-stranded part of a single-stranded/double-stranded DNA junction and RNA junction surprisingly stands vertically on graphene represents the central structural element of this invention. Specifically, it has been shown that double-stranded DNA and RNA adopts a vertical orientation when immobilized on graphene by a single-stranded toehold (see FIG. 1 and FIG. 6). For example, to achieve the immobilization of the dsDNA, the construct shown in FIG. 1a has been used, where the ssDNA segment is responsible for anchoring the whole molecule to the surface through IT-stacking interactions. The vertical immobilization of double-stranded DNA with a single-stranded DNA extension on graphene as described herein can only be explained if the lowest double-stranded nucleotide is firmly attached to the graphene which is considered to be the key structural feature of this invention. Above this firm attachment point, the DNA behaves like typical B-DNA. Only this firm orientation of the DNA enables resolution of down to a single nucleotide (<0.34 nm) that could not be achieved for a DNA wobbling in the repulsion field of a negatively charged surface.

The invention as illustrated in the appended examples, thus, offers various potential applications. A dye at the upper end of the vertical nucleic acid, for example, represents an excellent reporter of structural changes occurring to the nucleic acid below. Bending, bulges and nucleic acid distortion could directly be visualized and translated into specific structural features. Beyond, dye-labeled proteins interacting with the nucleic acid could be visualized while travelling along the DNA, in search mechanisms and in DNA processing with down to single nucleotide resolution. Thus, the approach enables functional assays of important proteins such as transcription factors and repair enzymes. Compared to existing methods such as FRET (Förster resonance energy transfer) and single-molecule FRET (smFRET), GETvNA offers advantages such as a simpler immobilization protocol, less noise from fluorophores exploring accessible volumes, and an unbleachable, spectrally broadband, non-blinking acceptor. Compared to smFRET, the attachment of the DNA to the surface is simple and does not involve the presence of chemical linkers. Since only one donor dye is needed, the noise introduced by fluorophores exploring an accessible volume is reduced compared to FRET, where two dyes are simultaneously attached to DNA. As only one color is required, further color channels are unnecessary or could be used further for complementary information. Moreover, unspecific binding of dyes to the surface is accompanied by strong quenching so that the background is not increased. With respect to DNA bending, smFRET lacks accuracy, since rotations and torsions that can accompany the bending directly affect the FRET outcome, while this does not have an influence on the GETvNA angle determinations. Therefore, GETvNA could retrieve actual bending angles and be used to study dynamic changes due to the binding of proteins with high temporal resolution. On the other hand, GETvNA experiments involving proteins diffusing along DNA can be followed with high sensitivity in a distance range that is 5-fold larger when compared to smFRET. In addition to that, GETvNA can be used to electrically contact DNA, to produce chips of single layers of dsDNA standing on graphene capable of holding functionalities or structures at well-defined distances from the surface.

Overall, GETvNA is a simple, inexpensive, and robust approach that offers greater precision and accuracy than current methods for studying nucleic acids as DNA or RNA, as well as, for example, DNA-protein interactions. Moreover, it has the potential to serve as the foundation for biosensors based on vertically oriented DNA chips. As also RNA is taking the vertical orientation, GETVNA can help to reveal the structures of various functional RNAs such as ribozymes that are difficult to resolve by other techniques and that may present multiple biologically important states. GETvNA might also help to reveal dynamic structural features within RNA as well as their interaction with partners such as proteins.

Thus, the present invention relates to a construct comprising a graphene substrate and a hybrid molecule. It is understood that the term “comprising”, as used throughout this description, denotes that further sequences, components and/or steps (e.g., when describing a method) can be included in addition to the specifically recited sequences, components and/or steps. However, this term also encompasses that the described subject-matter consists of exactly the recited sequences, components and/or method steps. The term “consisting of” only includes specifically mentioned features but does not include optional unspecified ones. Accordingly, the term “construct” as used herein refers to a conceptual element composed of at least two single elements, i.e., a graphene substrate and a hybrid molecule as defined herein.

In the present invention, the term “graphene substrate” refers to a substrate comprising graphene, which is a two-dimensional material composed of a monoatomic crystalline sheet of carbon with conjugated π electrons. Accordingly, the term “graphene” is understood in the context of the present invention as referring to a pristine 2D hexagonal carbon lattice. It is considered that the term “graphene” does not refer to non-pristine graphene with functional groups, graphene oxide (GO) being mentioned as an example. As explained in the introduction section, the results reported in the Piao publication (Piao Y, et al., Chemical Communications, 2011, 47:12149-12151) prove that the use of graphene oxide would not lead to a vertical orientation of the DNA. In the context of the present invention, graphene comprised in the graphene substrate can be provided as graphene monolayer or multiple graphene layers such as bi- or tri-layers. A skilled person knows how to optimize the number of graphene layers, considering that about 2.3% of the light is absorbed with each graphene layer which decreases the signal-to-noise ratio. In a preferred embodiment, graphene is located at the top of the graphene substrate, i.e., provides the surface in contact with the solution (e.g., comprising the hybrid molecule) to be reacted with. In a preferred embodiment, the graphene substrate further comprises an additional matrix different from graphene. The term “matrix” as used in the context of the invention refers to an optional basal element of the graphene substrate. Accordingly, the skilled person will appreciate that the matrix provides the base of the graphene substrate while graphene is immobilized on top of the matrix. The matrix can be a non-transparent material or a transparent material, while a transparent matrix is preferred. Suitable examples of the non-transparent matrix include silicon, and suitable examples of the transparent matrix include glass, polymers, sapphire, indium tin oxide, and fused silica. In the present invention, the matrix is preferably glass, even more preferably borosilicate glass A specific example thereof includes cover glass made from borosilicate glass with thickness of about 170 μm, however, the invention is not restricted thereto.

The term “about” as used herein means that the value may vary ±10%.

The term “hybrid molecule” as used herein refers to a molecule comprising a single-stranded nucleic acid segment and a double-stranded nucleic acid segment. As such, the hybrid molecule comprises a segment consisting of a single strand of a nucleic acid sequence and a segment consisting of a double strand of a nucleic acid sequence, wherein the single strand of the nucleic acid sequence forms one strand of the double strand nucleic acid sequence. Accordingly, they form a single molecule in which the maximum number of nucleotides/bases is not particularly limited. The base where the single-stranded nucleic acid segment joins the double-stranded nucleic acid segment is herein referred to as single-stranded nucleic acid/double-stranded nucleic acid junction.

The term “nucleic acid sequence” relates to a sequence of polynucleotides/nucleic acid molecules comprising purine- and pyrimidine bases. Thus, in the context of the invention, the terms “nucleic acid” includes DNA, such as cDNA, genomic DNA, synthetic forms of DNA, synthetic forms of DNA derivatives such as locked DNA, PNA, as well as RNA and mixed polymers comprising two or more of these molecules. It is understood that the term “RNA” as used herein comprises all forms of RNA including mRNA, tRNA and rRNA but also genomic RNA, such as in case of RNA of RNA viruses. The single-stranded nucleic acid segment and the double-stranded nucleic acid segment, may, thus, be a single-stranded DNA (ssDNA) segment and a double-stranded DNA (dsDNA) segment, or be a single-stranded RNA (ssRNA) segment and a double-stranded RNA (dsRNA) segment, as well as mixtures thereof. In one embodiment, it is imagined that the junction at the graphene substrate is formed by the DNA, but a large fraction of the double-stranded part is formed by RNA. This can be realized either by synthetic DNA/RNA hybrid or by hybridization of DNA with RNA prior to immobilization on graphene. In the present invention, it is preferred that the single-stranded nucleic acid segment is a ssDNA segment, and the double-stranded nucleic acid segment is a dsDNA segment. Exemplary nucleic acid sequence forming exemplary hybrid molecules of the invention are depicted in SEQ ID NO: 1 to SEQ ID NO: 18 and are further referred to in the Examples. The sequences reported in the sequence listing are exemplarily mentioned for illustration purposes only. The invention should not be understood in such a way that the embodiments described herein are limited to these particular sequences.

The nucleic acid sequences may be of natural as well as of synthetic or semi-synthetic origin. Thus, the nucleic acid molecules may, for example, be nucleic acid molecules that have been synthesized according to conventional protocols of organic chemistry. The person skilled in the art is familiar with the preparation and the use of such nucleic acid molecules (see, e.g., Sambrook and Russel, “Molecular Cloning, A Laboratory Manual”, Cold Spring Harbor Laboratory, N.Y. (2001)). Accordingly, further included are nucleic acid mimicking molecules known in the art such as synthetic or semi-synthetic derivatives of DNA or RNA and mixed polymers, both sense and antisense strands. They may contain additional non-natural or derivatized nucleotide bases, as will be readily appreciated by those skilled in the art. Such nucleic acid mimicking molecules or nucleic acid derivatives according to the invention include peptide nucleic acid (PNA), phosphorothioate nucleic acid, phosphoramidate nucleic acid, 2′-O-methoxyethyl ribonucleic acid, morpholino nucleic acid, hexitol nucleic acid (HNA) and locked nucleic acid (LNA), an RNA derivative in which the ribose ring is constrained by a methylene linkage between the 2′-oxygen and the 4′-carbon (see, for example, Braasch and Corey, Chemistry & Biology, 2001, 8:1-7). PNA is a synthetic DNA-mimic with an amide backbone in place of the sugar-phosphate backbone of DNA or RNA, as described in, e.g., Nielsen, et al. (Science, 1991, 254:1497) and Egholm, et al. (Nature, 1993, 365:666). Furthermore, it is envisaged for further purposes that nucleic acid may contain, for example, thioester bonds and/or nucleotide analogues. Nucleic acid molecules can be designed and purchased commercially, e.g., from Eurofins Genomics, Integrated DNA Technologies, and Biomers) as is known in the art.

A skilled person will appreciate that the nucleic acid strands in the double-stranded nucleic acid segment need a minimum complementarity for the double-stranded nucleic acid segment duplex to be thermally stable. In the present invention, the term “complementary” refers to a nucleotide sequence that base-pairs by non-covalent bonds to all or a region of a second nucleotide sequence (e.g., a first single-stranded nucleic acid to a second single-stranded nucleic acid). In the canonical Watson-Crick base pairing, adenine (A) forms a base pair with thymine (T), as does guanine (G) with cytosine (C) in DNA. In RNA, thymine is replaced by uracil (U). As such, A is complementary to T and G is complementary to C. In RNA, A is complementary to U and vice versa. Typically, “complementary” refers to a nucleotide sequence that is at least partially complementary. In this case, a single-stranded nucleic acid sequence may be partially complementary to a second single-stranded nucleic acid sequence, i.e., in which not all nucleotides are complementary to every nucleotide in the second single-stranded nucleic acid sequence in all the corresponding positions. The term “complementary” may also encompass duplexes that are fully complementary such that every nucleotide in one strand is complementary to every nucleotide in the other strand in corresponding positions-provided that the first single-stranded nucleic acid and the second single-stranded nucleic acid form a single-stranded nucleic acid overhang so as to allow attachment of the single-stranded nucleic acid segment to the graphene substrate. For example, a single-stranded nucleic acid sequence may be perfectly (i.e., 100%) complementary to a part of a second single-stranded nucleic acid sequence so as to form a hybrid molecule of the invention having 100% sequence identity in the double-stranded nucleic acid segment. Alternatively, the two strands of the double-stranded nucleic acid segment may share some degree of complementarity which is less than perfect (e.g., above 70%, 75%, 85%, 90%, 95%, 99%). Standard methods to determine percent complementarity of two nucleotide sequences are known in the art. In the present invention, the degree of complementarity is not particularly limited, provided that the double-stranded nucleic acid segment comprised in the hybrid molecule is stable.

In the present invention, the hybrid molecule is immobilized to the graphene substrate via the single-stranded nucleic acid segment. The term “immobilized” is understood as to refer to the natural adsorption of single-stranded nucleic acid such as ssDNA to graphene via non-covalent TT-stacking interactions. A double-stranded nucleic acid such as a dsDNA molecule does not adsorb because its bases are compromised in base pairing forming the double helix. The single-stranded nucleic acid is at least 5 nucleotides in length, as stability of immobilisation of the single-stranded nucleic acid to the graphene substrate is thought to decrease when having less than 5 nucleotides in the single-stranded nucleic acid segment. Preferably, the single-stranded nucleic acid segment is between 5 and 500, more preferably between 5 and 250, even more preferably between 5 and 100, and most preferably between 40 and 100 nucleotides in length. It should be understood that the present invention does not exclude the presence of a second single-stranded nucleic acid in the hybrid molecule. That is, the double-stranded nucleic acid segment is not considered to be restricted to have blunt ends. A skilled person will realize that the double-stranded nucleic acid segment may extend into a short single-stranded overhang provided that the single-stranded region does not interact with, i.e., immobilize to, the graphene substrate. Determining the maximal number of nucleotides in such a second single-stranded overhang so as to avoid attachment to the graphene substrate is routine in the art. In such an orientation of the hybrid molecule, the photoluminescent particle as defined herein may be attached to the second single-stranded nucleic acid overhang. In the context of the present invention, the photoluminescent particle may also bind to the double-strand nucleic acid segment as further explained herein below.

In the present invention, the double-stranded nucleic acid segment comprises a linear segment having at least one base pair in perpendicular orientation to the graphene substrate, which base pair is the first base pair extending from the single-stranded nucleic acid/double-stranded nucleic acid junction. The term “perpendicular” orientation as used in the present invention can be used interchangeably with “vertical” or “upright” and describes a 90° angle of the linear segment to the surface of the graphene substrate. In the present invention, the “first base pair” of the double-stranded nucleic acid segment, i.e., the first base pair extending from the single-stranded nucleic acid/double-stranded nucleic acid junction, is the first base pair of the linear segment. The term “linear segment” is understood herein as double-stranded nucleic acid sequence that extends straight from the first base pair without bending. Accordingly, in case the linear segment comprises more than the at least one (i.e., the first) base pair which is in perpendicular orientation to the graphene substrate, e.g., two, three, four, or more base pairs, these remaining base pairs comprised in the linear segment are also arranged in perpendicular orientation to the graphene substrate. The maximal number of base pairs in the linear segment is not particularly limited.

It is preferred that the first base pair at the single-stranded nucleic acid/double-stranded nucleic acid junction is immobilized with respect to its angle to the graphene substrate, i.e., naturally adsorbed, to the graphene substrate. A minimum distance of 5 nm between a photoluminescent particle and the graphene substrate is needed to have a readable fluorescence signal. For single-layer graphene, no quenching is detectable at 40 nm or higher. In the present invention, the distance z between a photoluminescent particle and the graphene substrate can be determined by using Equation 1:

$$z = {d}_{0} * {( \frac{1}{1 - \frac{τ}{{τ}_{0}}} - 1 )}^{\frac{1}{4}} \qquad ( 1 )$$

In this equation, τ represents the luminescence lifetime of a photoluminescent particle at a given distance to graphene, τ₀ is the emission lifetime in the absence of graphene, and do is the distance of 50% quenching efficiency. Because a minimum distance of 5 nm between a photoluminescent particle and the graphene substrate is needed to have a readable fluorescence signal, the minimum length of the linear segment including the first base pair at the single-stranded nucleic acid/double-stranded nucleic acid junction is considered to be between 15 and 20 base pairs. The skilled person is aware that each determination method is subject to measuring inaccuracy. In the present experimental setting, when measuring a distance of 10 nm with a precision of around 0.5 nm, the expected real distance will be between 9.5 and 10.5 nm. Accordingly, the skilled person understands that the exact minimum length of the linear segment is dependent on signal-to-noise and time resolution. In the present invention, the linear segment comprised in the double-stranded nucleic acid segment is at least 15 base pairs, preferably at least 20 base pairs, more preferably at least 36 base pairs long.

In general, the angle between the double-stranded nucleic acid segment and the graphene substrate is referred to herein as angle alpha (α). Alpha (α) can be calculated by using Equation 2:

$$a = arcsin ( \frac{z}{L} ) \qquad ( 2 )$$

In this equation, L represents the length of the double-stranded nucleic acid fragment and z the distance between a photoluminescent particle and the graphene substrate. Since Equation 2 has no solution for z>L, z values exceeding L are computed as if they are equal to L. In the present invention, the double-stranded nucleic acid segment comprising a linear segment having at least one base pair in perpendicular orientation to the graphene substrate has an angle alpha (α) of 90°.

In the present invention, the double-stranded nucleic acid segment as comprised in the construct of the invention may comprise at least one bend. Thus, the skilled person understands that the double-stranded nucleic acid segment of the invention may comprise one or more, e.g., two, three, four or more bends. The number of bends as comprised in the double-stranded nucleic acid segment is not particularly limited. In the present invention, it is preferred that the double-stranded nucleic acid segment as comprised in the construct of the invention comprises one bend. A bent nucleic acid sequence is curved over a stretch of several bases, resulting in a bending angle divergent from the 90° angle of the above-described perpendicular linear segment as comprised in the double-stranded nucleic acid segment of the invention to the graphene substrate. The angle resulting from bending is herein referred to as kink angle θ. In the present invention, it is considered that a bend as comprised in the double-stranded nucleic acid segment is followed by a linear nucleic acid segment which extends from the bend and has a non-perpendicular orientation to the graphene substrate. Accordingly, the linear segment which has a non-perpendicular orientation to the graphene substrate extends straight with no bending following the given kink angle θ. The kink angle θ can be calculated from the height of the dye through Equation 3:

$$θ = arcsin ( \frac{z - {z}_{kink}}{L - {z}_{kink}} ) \qquad ( 3 )$$

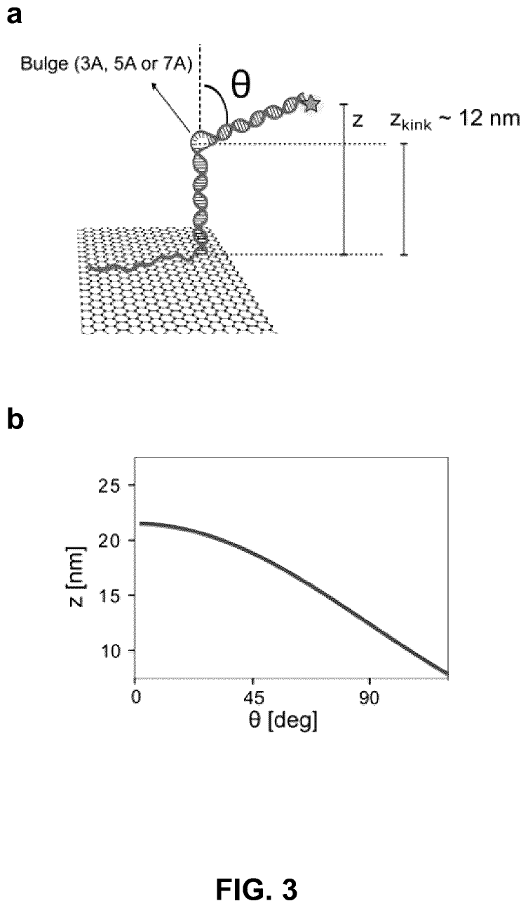

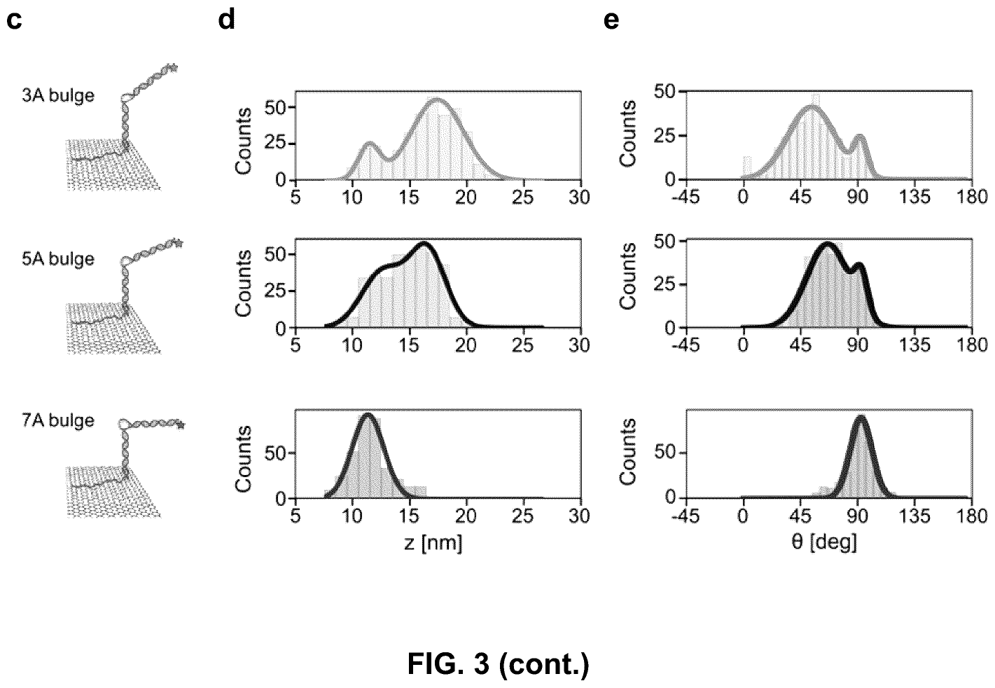

z refers to the total distance between the dye and graphene and zₖᵢₙₖ refers to the distance between graphene and the bend when the bend is geometrically collapsed into a single point. In other words, in many cases (e.g., for bulges and Endonuclease IV binding) bending can be assumed to be point-like with little approximation, when this is not the case (e.g., for A-tract bending), the bending was considered to be located in the middle of the sequence. z and zₖᵢₙₖ are visualized in FIG. 3a, L depicts the length of the double-stranded nucleic acid segment. In the present invention, the number of bases in the linear segment which extends from the bend and has a non-perpendicular orientation to the graphene substrate is not particularly limited. However, the skilled person knows that the linear segment needs to comprise a minimum number of complementary nucleotides in order to show a stable hybridization. As such, determining the minimum number of bases in the linear segment is routine in the art.

The skilled person is aware that bending of nucleic acids, e.g., DNA, may occur in response to several events including, but not limited to, the presence of unpaired mismatches, abasic (AP) sites, A-tracts, nicks, or binding of different proteins that interact with nucleic acids (Bates A D and Maxwell A, Oxford University Press, 2005, ISBN: 9780198506553; Sinden R R, Academic Press, 1994, ISBN 9780080571737; Dickerson R E, Nucleic Acids Research, 1998, 26:1906-1926; Koo H S. et al., Nature, 1986, 320:501-506; Wu H M and Crothers D M, Nature, 1984, 308:509-513; Nadeau J G and Crothers D M, Proceedings of the National Academy of Sciences, 1989, 86: 2622-2626; Thompson J F and Landy A, Nucleic Acids Research, 1988, 16:9687-9705; Banfalvi G, Biochemistry and Molecular Biology Education, 1994, 22:104-104; Velmurugu Y, Springer, 2016, ISBN: 9783319451299; Calladine C, et al., Academic Press, 2004, ISBN: 9780080474663). In the present invention, the bend preferably originates from the presence of unpaired adenine bulges or nucleic acid-interacting proteins. The nucleic acid-interacting proteins are not particularly limited as long as they effect bending of the nucleic acid sequence. Examples thereof include polymerases, single-stranded binding proteins, histones and restriction enzymes. A preferred example of a nucleic acid-interacting protein is as mutant Escherichia coli endonuclease IV (Protein Data Bank ID 2NQJ, released: Nov. 6, 2007, published by Garcin, et al., Nature Structural & Molecular Biology, 2008, 15:515-522).

In a preferred embodiment, the double-stranded nucleic acid segment comprises at least one photoluminescent particle. The term “photoluminescent particle” as used herein is understood as to refer to compounds that emit light after excitation. Photoluminescent particles are well-known in the art, quantum dots, fluorescent beads and fluorescence dyes (fluorophores) being mentioned as preferred examples. In the context of the present invention, the photoluminescent particle is preferably a quantum dot or a fluorophore, most preferably a fluorophore. Suitable examples of fluorophores include AlexaFluor dyes (e.g., Alexa Fluor 647 or Alexa Fluor 568), ATTO dyes (e.g., ATTO647N, ATTO542, ATTO643 or ATTO532), Janelia Fluor dyes (Janelia Fluor 549, Janelia Fluor 635 or Janelia Fluor 646) and Cyanines (e.g., Cy3B, Cy3, Cy5B or Cy5), and Rhodamines (e.g., TAMRA, Rhodamine6G or Rhodamine110). Suitable quantum dots include CdSe/CdZnS core-shell quantum dots and PbS colloidal quantum dots. However, the skilled person appreciates that the present invention is not limited to a particular type of photoluminescent particle, e.g., a particular type of fluorophore or quantum dot.

In the present invention, the total number of photoluminescent particles as comprised by the double-stranded nucleic acid segment is not particularly limited. As such, it is considered that the double-stranded nucleic acid segment may comprise several photoluminescent particles, of one type or of mixed types. In a non-limiting example, the double-stranded nucleic acid segment may comprise a mixture of several fluorophores and/or several quantum dots, so that one could imagine having dyes of different color in order to monitor different distances to the graphene substrate in parallel.

The “at least one photoluminescent particle” as comprised by the double-stranded nucleic acid segment may be directly attached to the double-stranded nucleic acid segment. In the present invention, the term “directly” attached” refers to the direct attachment of the photoluminescent particles to the nucleic acid, e.g., directly attached to one base nucleotide.

Alternatively, the at least one photoluminescent particle as comprised by the double-stranded nucleic acid segment may be indirectly attached to the double-stranded nucleic acid segment. In the present invention, the term “indirectly” attached” refers to the indirect attachment of the photoluminescent particles to the nucleic acid via a protein bound to the double-stranded nucleic acid segment. In other words, indirect attachment is achieved by labeling a nucleic acid-binding protein with a photoluminescent particle as described herein (Obermaier C, et al., Principles of Protein Labeling Techniques. In: Posch A. (ed.) Proteomic Profiling. Methods in Molecular Biology, 2015, 1295. Humana Press, New York, NY; ISBN 9781493925490; Further, Resource Libraries https://www.thermofisher.com/de/de/home/life-science/protein-biology/protein-biology-learning-center/protein-biology-resource-library/pierce-protein-methods/methods-labeling-nucleic-acids.html and https://international.neb.com/products/dna-modifying-enzymes-and-cloning-technologies/dna-labeling/dna-labeling). DNA-binding proteins are well-known in the art and the present invention is not particularly limited in this regard (Stryer L, et al., Macmillan Publishing, 2019, Ninth edition, ISBN: 9781319234362). A preferred DNA-binding protein is 06-alkylguanine DNA alkyltransferase (AGT) which has been exemplary tested in the examples. AGT is a protein involved in repairing alkylguanine lesions that can be highly mutagenic and cytotoxic. In a preferred embodiment of the invention, the at least one photoluminescent particle as comprised by the double-stranded nucleic acid segment which is directly or indirectly attached to the double-stranded nucleic acid segment is a fluorophore or a quantum dot.

The at least one photoluminescent particle as comprised by the double-stranded nucleic acid segment, which may be directly or indirectly attached to the double-stranded nucleic acid segment, may be attached to any base of the nucleic acid. Accordingly, the position of attachment to the nucleic acid is not particularly limited and can be, for example, at the last base pair, at any internal base pair, or, in case of multiple photoluminescent particles, at multiple positions thereof. In one embodiment, the at least one photoluminescent particle is directly attached to the last base pair of the double-strand DNA segment extending from the junction of the single-stranded nucleic acid segment and the double-stranded nucleic acid segment.

The present invention further relates to a method for producing the construct of the invention. The method comprises the step of mixing a graphene substrate as disclosed herein and a hybrid molecule comprising a single-stranded nucleic acid segment and a double-stranded nucleic acid segment as disclosed herein to allow immobilization of the single-stranded nucleic acid segment to the graphene substrate. Alternatively, the method comprises the step of adding a first single-stranded nucleic acid to a graphene substrate as disclosed herein to allow attachment of the first single-stranded nucleic acid to the graphene substrate, and adding a second single-stranded nucleic acid that is complementary to the first single-stranded nucleic acid to form a hybrid molecule as disclosed herein comprising a single-stranded nucleic acid segment and a double-stranded nucleic acid segment. The skilled person will appreciate that in order to form a hybrid molecule in the context of the method for producing the construct, the first single-stranded nucleic acid and the second single-stranded nucleic acid must form a single-stranded nucleic acid overhang so as to allow attachment of the single-stranded nucleic acid segment to the graphene substrate. In the present invention, it is preferred that the method comprises the step of mixing a graphene substrate as disclosed herein and a hybrid molecule as disclosed herein to allow immobilization of the single-stranded nucleic acid segment to the graphene substrate.

Suitable experimental conditions for producing the construct can routinely be determined and are further exemplified in the working examples described herein. Specifically, a person skilled in the art is aware that adequate salt concentrations are required when working with hybridized nucleic acids as without salts they would fall apart due to coulombic repulsion. Salts which keep hybridized nucleic acids stable are known in the art, NaCl being mentioned as an example. A skilled person will appreciate that such salts do not affect the determination of the fluorescence intensity nor for the fluorescence lifetime. In a preferred embodiment, the construct is produced with 500 mM NaCl buffer or 50 mM NaCl buffer, dependent on the experimental setting.

In a preferred embodiment, the construct as obtainable by the production method of the invention is characterized by having a double-stranded nucleic acid segment comprising a linear segment comprising at least one base pair in perpendicular orientation to the graphene substrate at the junction where the double-stranded nucleic acid segment and the single-stranded nucleic acid segment join. Accordingly, the construct as obtainable by the method of the invention for producing the construct is the construct as described in embodiments disclosed herein. The skilled person understands that the present invention encompasses the construct comprising a graphene substrate and a hybrid molecule comprising a single-stranded nucleic acid segment and a double-stranded nucleic acid segment as obtainable by the production method disclosed herein.

The method for producing the construct of the invention as disclosed herein may optionally comprise a step of (i) attaching at least one photoluminescent particle to the single-stranded nucleic acid forming the double-stranded nucleic segment, or (ii) attaching at least one photoluminescent particle to the double-stranded DNA segment. As described throughout the description, attachment includes direct attachment and indirect attachment as defined herein. The time point of attaching the at least one photoluminescent particle is not particularly limited, provided that the construct as produced by the production method of the invention comprises at least one photoluminescent particle in the double-stranded nucleic acid segment as described herein. In non-limiting examples, attaching the at least one photoluminescent particle may be performed before or after mixing a graphene substrate as disclosed herein and a hybrid molecule as disclosed herein, or before or after adding a first single-stranded nucleic acid as disclosed herein to a graphene substrate as disclosed herein, or before or after adding a second single-stranded nucleic acid that is complementary to the first single-stranded nucleic acid attached to the graphene substrate to form a hybrid molecule as disclosed herein. In the present invention, it is preferred that the method comprises the step of mixing a graphene substrate as disclosed herein and a hybrid molecule as disclosed herein, wherein the double-stranded nucleic acid segment is labeled with at least one photoluminescent particle before mixing with the graphene substrate. Methods to attach at least one photoluminescent particle to, i.e., label, a nucleic acid sequence at a desired base position are routine in the art. Labeling occurs, e.g., by phosphoamidite chemistry, or to amino modified nucleotides, e.g., by NHS chemistry or to thiol modified nucleotides, e.g., by maleimide chemistry or to biotin modified nucleotides through avidin or by click chemistry.

The present invention further relates to the use of the construct of the invention. The skilled person will appreciate that the invention offers various potential applications. In exemplary embodiments, the use of the construct of the invention is for reporting structural changes occurring to the nucleic acids, for visualizing bending of nucleic acids, bulges in nucleic acids as well as nucleic acid distortion, for visualizing dye-labeled proteins interacting with (e.g., travelling along) a nucleic acid, for producing chips of single layers of dsDNA standing on graphene. Deformation of the nucleic acid can also be used to detect and determine the concentration of analyte molecules such as transcription factors or DNA repair proteins.

In a preferred embodiment, the invention relates to the use of the construct as described herein for measuring fluorescence lifetime or fluorescence intensity. The skilled person will appreciate that the specific use of measuring fluorescence lifetime or fluorescence intensity requires the construct of the invention to comprise at least one photoluminescent particle as described herein.

Accordingly, in a specific embodiment, the invention relates to the use of the construct wherein the double-stranded nucleic acid segment comprises at least one photoluminescent particle, or the construct as obtainable by the production method described herein, wherein the double-stranded nucleic acid segment comprises at least one photoluminescent particle.

The term “fluorescence lifetime” as used herein refers to the time between the emission of a photon by a photoluminescent particle after it was excited by a short laser pulse. The term “fluorescence intensity” refers to the signal intensity (number of photons) detected from a photoluminescent particle. A skilled person appreciates that the fluorescence lifetime and fluorescence intensity as used in the present invention are proportional to each other. It is considered that fluorescence lifetime readout can be advantageous as it is independent of excitation intensity and number of molecules within one spot. The term “quenching” as used herein describes the shortening of the fluorescence lifetime and the reduced fluorescence intensity by competing energy transfer to graphene. The above-defined principles are common-general knowledge in the art (Lakowicz J R, Springer-Verlag US, 2006, ISBN 978-0-387-31278-1).

The present invention further relates to a method for measuring quenching efficiency. Specifically, the method is characterized by measuring quenching efficiency by means of measuring the fluorescence lifetime and/or fluorescence intensity in a construct of the invention or in a construct as obtainable by the production method of the invention. Quenching efficiency refers to the reduction of the fluorescence intensity and of the fluorescence lifetime by energy transfer to graphene. The quantum yield is determined by the ratio of the radiative rate constant divided by the sum of all rate constants depopulating the excited state. Energy transfer to graphene is an additional rate constant depopulation the excited state of the dye. The quenching efficiency refers to fraction of excited state visits that is depopulated by energy transfer to graphene (Lakowicz J R, Springer-Verlag US, 2006, ISBN 978-0-387-31278-1). Means for measuring quenching efficiency by means of measuring the fluorescence lifetime and/or the fluorescence intensity are known to a skilled person and are not particularly limited. A standard device for measuring the fluorescence intensity is a fluorescence spectrometer. A standard device for measuring the fluorescence lifetime is a fluorescence spectrometer with functionality for time-correlated single-photon-counting. Both can also be done appropriately equipped fluorescence microscopes. In a preferred embodiment, quenching efficiency by means of measuring the fluorescence lifetime or fluorescence intensity is measured by using Fluorescence Lifetime Imaging Microscopy. As explained above, a skilled person is aware that Fluorescence Lifetime Imaging Microscopy records both fluorescence intensity and fluorescence lifetime. In a non-limiting example, fluorescence lifetime and/or fluorescence intensity can be determined by using the microscopy system of the invention.

The present invention further relates to a microscopy system for measuring fluorescence lifetime in a construct as described herein and comprising a photoluminescent particle, the construct comprising a graphene substrate and a hybrid molecule comprising a single-stranded nucleic acid segment and a double-stranded nucleic acid segment, wherein the hybrid molecule is immobilized to the graphene substrate by the single-stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join. Particularly, the microscopy system may comprise: an excitation path, an objective, a detection path, a detector, and a Time Correlated Single Photon Counting unit; wherein the excitation path comprises a laser and is configured to produce a pulsed laser beam and direct the pulsed laser beam into the objective using one or more beam redirecting elements, the pulsed laser beam having a pulse width below 750 ps, preferably below 500 ps, more preferably below 200 ps; wherein the objective is configured to transmit the pulsed laser beam into a sample comprising the construct and to collect light from the sample, particularly light emitted from the construct; wherein the detection path is configured to direct light from the objective, particularly the light from the sample, to the detector using one or more beam redirecting elements; wherein the detector is configured to detect the light received from the detection path, particularly the light from the sample that is directed onto, e.g., focused on, the detector using a lens; and wherein the microscopy system is configured to measure an arrival time of individual photons at the detector after a laser pulse, using the Time Correlated Single Photon Counting unit.

The microscopy system may be configured to mount the sample such that a focal plane of the objective is located within the sample. For example, the microscopy system may comprise a stage for that purpose. The excitation path may be configured to shape the laser beam exiting the laser according to measurement requirements. A pulsed laser beam as used herein may comprise and/or consist of any number of pulses. For example, a laser beam may consist of a single pulse, two pulses, three pulses, or a higher number of pulses. As known in the art, when travelling along the detection path and/or the objective, the laser beam may be subject to intentional changes and/or unintentional changes such as intensity losses etc. The objective may comprise one or more lenses. Typically, the objective comprises several lenses. The beam redirecting elements may be of any kind known in the art. Particularly, the beam redirecting elements may include one or more mirrors such as metallic mirrors and dichroic mirrors, one or more acousto-optical elements, one or more lenses, one or more beamspitters that can be polarizing or not, or beams can be redirected by the use of optical fibers, and combinations thereof. The excitation path and/or the detection path may comprise further optical elements, e.g., one or more irises or apertures, one or more filters, one or more beam combiners, e.g., dichroic mirrors, one or more beam splitters, e.g., dichroic mirrors, means for adjusting the average power in the laser beam, one or more shutters for blocking light travelling along the excitation path/detection path, and combinations thereof. For example, in some embodiments, the average power of the pulsed laser beam is 10 μW or less, preferably 6 μW or less, more preferably 4 μW or less at the position of the objective. The microscopy system may be configured to set the average power according to the requirements of an application. For example, the microscopy system may be set to deliver a laser beam with an average power at the location of the objective that results in non-saturating conditions for the excitation of the fluorophores used in the application. For example, 4 μW may be used with various fluorescent dyes in the UV, visible and IR spectral regions including fluorescent dyes from the classes of coumarines, rhodamines, cyanines, xanthenes, pyrenes, oxazines and pyronins.

The excitation path may include any suitable means for shaping the beam, for example one or more mirrors such as metallic mirrors and dichroic mirrors, one or more acousto-optical elements, one or more lenses, one or more irises or apertures, one or more beamspitters that can be polarizing or not, or beams can be redirected by the use of optical fibers, and combinations thereof. One or more elements that are used for beam shaping may also be used for beam redirection and vice versa. The detection path may be configured to focus the light from the objective into a point-like detector, preferably an avalanche photodiode detector. Alternatively, or additionally, the detection path may be configured to direct the light from the objective onto a camera configured for fluorescence lifetime imaging. The detection path may comprise any suitable means for shaping and/or redirecting the light. For example, it may include one or more mirrors such as metallic mirrors and dichroic mirrors, one or more acousto-optical elements, one or more lenses, and combinations thereof.

The microscopy system may be configured to control the polarisation of the laser beam and/or the light in the detection path. For example, the microscopy system may comprise one or more waveplates, for example quarter and/or half wave plate(s) for that purpose. The microscopy system may be configured to provide laser beams of different central wavelengths to the sample. For example, the microscopy system may comprise a first laser configured to emit a laser beam of a first central wavelength, and a second laser configured to emit a laser beam of a second central wavelength that differs from the first central wavelength. Everything described herein with reference to the excitation path also applies to excitation paths with two or more laser lines. The components of the microscopy system may be configured to transmit and reflect the wavelengths as appropriate. The microscopy system may be configured to combine the laser lines at one or more points in the excitation path. For example, the first and second laser beams may be combined at a certain point in the excitation path, e.g., via a dichroic mirror. For each laser beam that is combined with another beam one dichroic mirror may be used. Alternatively, or additionally, one or more of the lasers may be tuneable, i.e., be configured to emit laser beams of different central wavelengths. The detection path may be configured according to the laser lines of the excitation path. Particularly, the detection path may be configured to direct light with wavelengths according to fluorescence molecule emission spectra corresponding to the excitation wavelengths of the microscopy system to the detector(s). The detection path may split into several lines in accordance with the used fluorescence spectra (i.e., detection spectra).

The microscopy system may comprise hardware, e.g., one or more controllers, and/or software configured to control and/or operate one, some or all of the components of the microscopy system and/or the whole system. The hardware may include a PC, data acquisition card(s), input/output interface(s), and the like. The microscopy system comprises a Time Correlated Single Photon Counting unit (in short TCSPC unit) and is configured to measure an arrival time of individual photons at the detector(s) after a laser pulse, using the TCSPC unit. The detection path uses a photomultiplier tube, a photodiode, or a hybrid detector for photon detection.

An exemplary microscopy system according to the present invention that was used to perform single-molecule fluorescence lifetime measurements in a construct as described herein is provided in the following: A standard inverted confocal microscope (inverted microscope, Olympus IX71) was equipped with a Time-Correlated Single-Photon Counting (TCSPC) unit. A part of the excitation path and a part of the detection path were custom built and outside the microscope body. The optical setup used comprised an excitation path comprising two pulsed lasers (Picoquant, LDH-P-FA-530B and LDH-D-C-640), which were configured to emit a laser beam at a central wavelength of 532±3 nm and a laser beam at a central wavelength of 636 nm, respectively. The lasers were coupled into a single mode fiber (P3-488PM-FC, Thorlabs GmbH) to obtain a Gaussian beam profile and to perfectly overlay the two excitation beams. Circular polarized light is obtained by a linear polarizer (LPVISE 100-A, Thorlabs GmbH) and a quarter-wave plate (AQWP05M-600, Thorlabs GmbH). Excitation light was then directed into the back port of the microscope body of the Olympus IX71 microscope, and is directed to the objective by a dichroic mirror (zt532/640rpc, Chroma) in the microscope body and was then focused onto the sample via an oil immersion objective (UPLSAPO 100 XO, NA 1.40, Olympus Deutschland GmbH). The position of the sample was adjusted using a piezo stage (P-517.3CD, Physik Instrumente (PI) GmbH & Co. KG) and a controller (E-727.3CDA, Physik Instrumente (PI) GmbH & Co. KG). Light emitted from the sample, i.e. emission light, was collected by the objective and directed out of the microscope body via the Olympus IX71's side camera port. The emission light was separated from the excitation beam by the dichroic mirror in the microscope body (zt532/640rpc, Chroma) and focused onto a 50 μm diameter pinhole (Thorlabs GmbH). After the pinhole, the donor and acceptor signals are separated by a dichroic beamsplitter (640 LPXR, Chroma) into a green (Brightline HC582/75, AHF; RazorEdge LP 532, Semrock) and red (SP 750, AHF; RazorEdge LP 647, Semrock) detection channel. Emission light was focused via lenses (AC080-020-A-ML, Thorlabs) onto avalanche photodiodes (SPCM-AQRH-14-TR, Excelitas) and the signals were registered by a time-correlated single photon counting (TCSPC) unit (HydraHarp400, PicoQuant). The setup is controlled by a commercially available software package (SymPhoTime64, Picoquant GmbH). For FRET experiments, both lasers were operated at a 20 MHz repetition rate. The laser pulses were altered on the nanosecond timescale by a multichannel picosecond diode laser driver (PDL 828 “Sepia II”, PicoQuant GmbH) with an oscillator module (SOM 828, PicoQuant GmbH). Further data analysis was performed with self-written Python routines. The measurements could also be performed on a wide-field setup, either using intensity-based measurements or using fluorescence lifetime cameras, available commercially nowadays. Further details of the components of the used microscopy system as well as imaging aspects can be found in https://www.pnas.org/doi/suppl/10.1073/pnas.2211896120/suppl_file/pnas.22118961 20.sapp.pdf; Zähringer J, et al., Light: Science & Applications, 2023, 12:70; Krause S, et al., ACS Nano, 2021, 15:6430-6438; and Kamińska I, et al., Advanced Materials, 2021, 33: 2101099.

### EXAMPLES

### Example 1

The following example investigates the orientation of DNA on graphene. The present invention, called GETvNA (Graphene Energy Transfer with vertical Nucleic Acids), uses graphene to immobilize a hybrid molecule composed of single-stranded DNA (ssDNA) and double-stranded DNA (dsDNA) in a well-defined perpendicular orientation. The dsDNA segment contains a dye, allowing for sub-nanometer precision measurements of the distance between the dye and the surface by measuring the efficiency of the energy transfer from the dye molecule to graphene. This makes GETvNA a unique tool for studying conformational changes on dsDNA, especially when DNA-protein interactions take place.

To determine the orientation of DNA on graphene, it was measured how efficiently the excitation energy of single fluorophores located at the end of the dsDNA segment is transferred to graphene (FIG. 1a). Along with other research results, it has been established that graphene is a broadband fluorescence quencher with a d⁻⁴ distance dependence and 50% energy transfer efficiency at 18 nm. FIG. 1b provides a representative graph that shows the relationship between the energy transfer efficiency and the distance from the fluorophore to graphene. Furthermore, FIG. 1c shows the theoretical limit for the precision to determine the distance to graphene, which lies below 1 nm even with a low number of photons (500-1500).

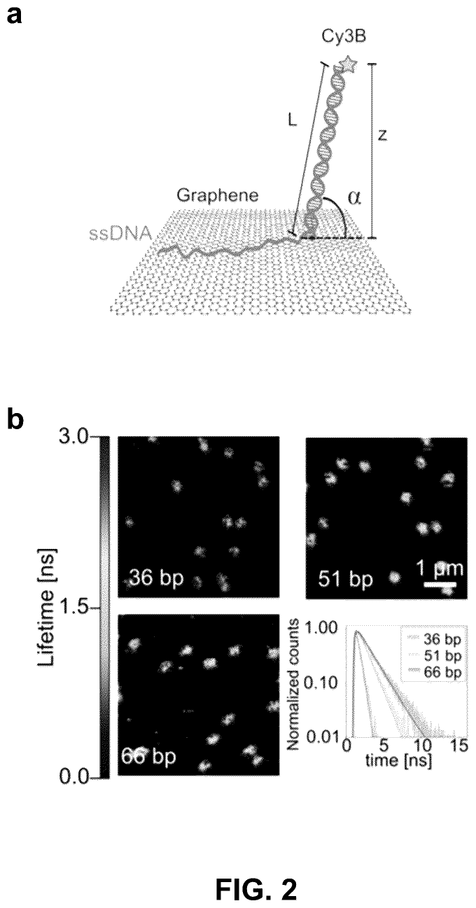

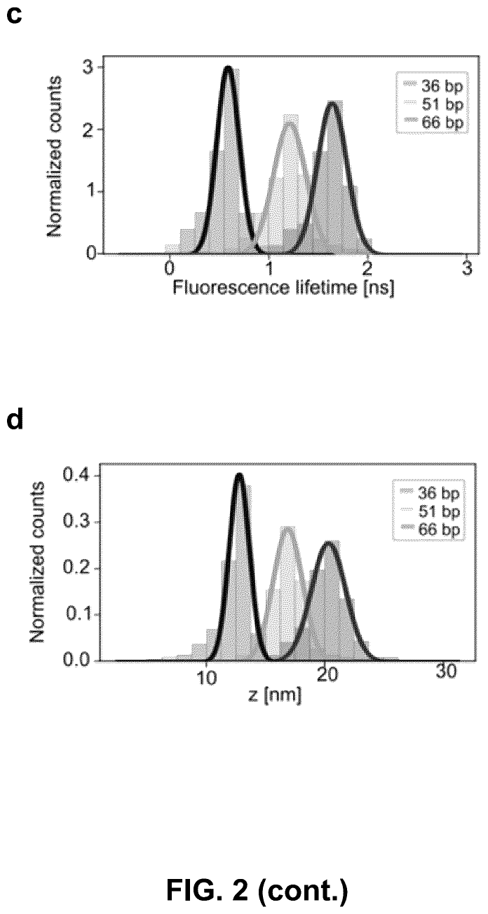

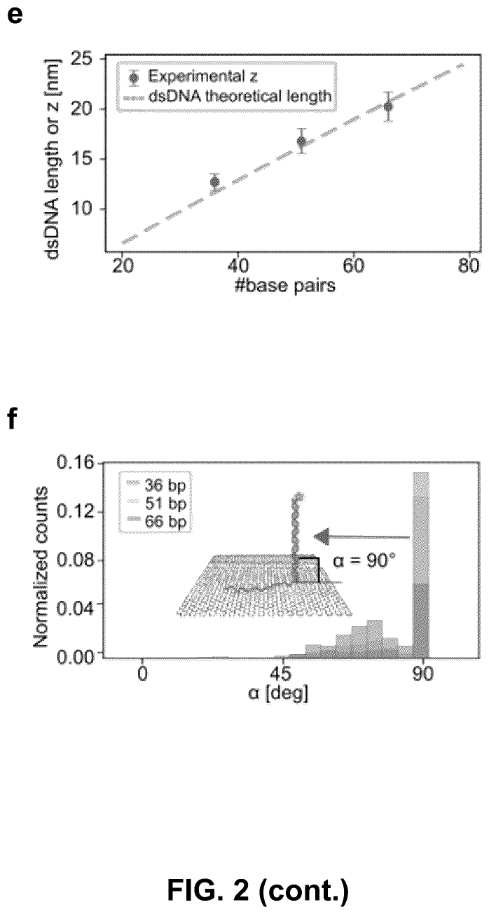

To investigate the actual orientation of the double helix, a fluorescent dye (Cy3B) was attached to one of its end bases, as depicted in FIG. 2a. Single-molecule fluorescence lifetime measurements were then performed and the distance between the dye and graphene, z, was obtained by using Equation 1:

$$z = {d}_{0} * {( \frac{1}{1 - \frac{τ}{{τ}_{0}}} - 1 )}^{\frac{1}{4}} \qquad ( 1 )$$

In this equation, τ represents the fluorescence lifetime of the fluorescent dye at a given distance to graphene, τ₀ is the fluorescence lifetime in the absence of graphene, and d₀ is the distance of 50% quenching efficiency. For Cy3B, calibration measurements were performed using DNA origami as nanopositioners and a d₀ value of (17.0±0.5) nm was obtained. Finally, knowing the length of the dsDNA fragment (L), it is possible to retrieve the angle between dsDNA and the graphene surface (α) by trigonometry and thus get their relative orientation.

To prepare the graphene substrate, an experimental procedure to obtain graphene-on-glass was used that is well-established (Krause S, et al., ACS Nano, 2021, 15:6430-6438). Starting from commercially available single-layer graphene grown by chemical vapor deposition (CVD) on a copper substrate, protected by a PMMA coating on top of graphene (ACS Material, CVD Graphene on Copper-PMMA Coated, SKU #CVCU1M02), the protocol consists of a first step of copper etching, followed by a transfer of graphene to a glass coverslip, and a last cleaning step where the PMMA is removed. The whole procedure involves inexpensive chemicals and easy manipulation of reagents. A detailed protocol can be found in multiple publications (Zähringer J, et al., Light: Science & Applications, 2023, 12:70; Krause S, et al., ACS Nano, 2021, 15:6430-6438; Kamińska I, et al., Advanced Materials, 2021, 33: 2101099).

To prepare the hybrid molecule, custom ssDNA was designed (Custom DNA Oligos in Tubes—Eurofins Genomics, Single-stranded DNA—Integrated DNA Technologies, Oligonucleotide—Biomers), and mixed to form the ssDNA-dsDNA hybrid molecule as described below. The mixing procedure was performed at 37° C. for two hours in TAE buffer (Tris—Acetate—EDTA, prepared from 10× concentrate Tris Acetate-EDTA buffer, Sigma Aldrich, SKU #T9650-1L) (pH=8.3) containing 500 mM NaCl. The ssDNA molecules were designed in order to (i) have one ssDNA longer than the other; (ii) completely hybridize the shortest of the ssDNA molecules with part of the longest ssDNA, forming this way the dsDNA segment; and (iii) have an extra ssDNA segment on the longest ssDNA, which represents the 5′ to 3′ overhang that is attached to the graphene surface. The shortest ssDNA was purchased already containing the fluorescent label (ssDNA-Cy3B, purchased from Eurofins) at the 5′ end. The hybrid molecule was then added to the graphene-on-glass sample in a 100 pM concentration at room temperature using the same 500 mM NaCl TAE buffer as described before. After 1 minute of incubation, the graphene sample was washed 3 times with the same 500 mM NaCl TAE buffer, to remove any excess of hybrid molecule that did not attach to graphene. ssDNA segments of lengths between 40 and 100 nucleotides can be used to pin the hybrid molecule system to graphene. While shorter strands could still work for this purpose, the stability of the immobilization will decrease when having less than 5 nucleotides. Finally, single-molecule lifetime measurements were carried out in a standard confocal microscope equipped with a Time-Correlated Single-Photon Counting (TCSPC) module. The optical setup used consisted of an excitation path comprising pulsed lasers (Picoquant, LDH-P-FA-530B and LDH-D-C-640). The laser beams were directed to the objective of the microscope using mirrors (Thorlabs, PF05-03-P01-Ø½″ Protected Silver Mirror), and the polarization of light was set as circular using quarter (Thorlabs, AQWP05M-600-Ø½″ Mounted Achromatic Quarter-Wave Plate, Ø1″ Mount, 400-800 nm) and half waveplates (Thorlabs, AHWP10M-600-Ø1″ Mounted Achromatic Half-Wave Plate, SM1-Threaded Mount, 400-800 nm). Excitation light was then directed to a commercial microscope body (inverted microscope, Olympus IX71), to finally pass through a dichroic mirror (zt532/640rpc, Chroma) and be focused onto the sample via an oil immersion objective (UPLSAPO 100 XO, NA 1.40, Olympus). The sample was scanned with a piezo-stage (P527.3CD, Physik Instrumente) which was controlled by an E-727 controller (Physik Instrumente). The light emitted from the sample was directed and focused via lenses (AC080-020-A-ML, Thorlabs) into an avalanche photodiode (APD, SPCM-AQRH-TR-14, Excelitas). The setup counted with a TCSPC unit (Time Correlated Single Photon Counting), which processes the APD's signal and allows the precise timing of photon arrival (in the setup, a Picoquant TCSPC system was used: HydraHarp 400, PicoQuant GmbH, Germany). Acquisitions were controlled with the software SymPhoTime 64 (PicoQuant). Further data analysis is performed with self-written Python routines. The measurements could also be performed on a wide-field setup, either using intensity-based measurements or using fluorescence lifetime cameras, available commercially nowadays. Further details of the components of the used microscope can be found in Zähringer J, et al., Light: Science & Applications, 2023, 12:70; Krause S, et al., ACS Nano, 2021, 15:6430-6438; and Kamińska I, et al., Advanced Materials, 2021, 33: 2101099.

dsDNA molecules of three different lengths 36, 51, and 66 base pairs (bp) were studied. To obtain these samples, different ssDNA molecules were combined to hybrid molecules. In all cases, the same 136 nucleotides long ssDNA was used (SEQ ID NO: 1), which was hybridized with three different ssDNA with lengths of 36 (SEQ ID NO: 2), 51 (SEQ ID NO: 3) and 66 (SEQ ID NO: 4) nucleotides (see Table 1). As a result, the obtained hybrid molecules contained dsDNA segments of 36, 51 and 66 base pairs and ssDNA overhangs of 100, 85 and 70 nucleotides, respectively. In FIG. 2b, exemplary FLIM (Fluorescence Lifetime Imaging Microscopy) images acquired for each sample are shown, where homogeneous lifetime values can be observed. The FLIM images were obtained using Sympho Time software (Picoquant) and using an average laser power of 4 μW (values between 1 and 10 μW are compatible with these measurements). The pixel size of the scan was set between 50 and 100 nm, and the dwell time (time spent in each pixel) between 0.5 and 2 ms. Next, to maximize the number of detected photons per molecule, time traces were measured and fluorescence lifetime decays for each molecule were obtained. As depicted in the examples from the fourth panel of FIG. 2b, the decays were monoexponential for all three samples. Therefore, fluorescence lifetime values for every single molecule were obtained through monoexponential fits, and their distances to graphene were calculated by Equation 1. In FIGS. 2c and 2d, the histograms of fluorescence lifetime and distance to graphene are shown.

The experimental heights of the dyes retrieved from the Gaussian fits were then overlaid with the theoretical lengths of dsDNA, which were calculated using the Worm-Like chain model. The resulting plot is displayed in FIG. 2e and shows that the height of the end of the dsDNA coincides with the length of the double helix, which can only be explained if the dsDNA stands perpendicular to the surface. It also implies that the lowest double stranded nucleotide must have a fixed orientation on graphene. Otherwise, the wobbling would lead to lower average distances of the dye to the graphene. This is a key structural element of the invention, that is the surprising finding that the lowest base pair shows no orientational fluctuations with respect to graphene.

To further investigate the latter, a was calculated using Equation 2:

$$a = arcsin ( \frac{z}{L} ) \qquad ( 2 )$$

Since this equation has no solution for z>L, z values exceeding L were computed as if they were equal to L. From the histograms shown in FIG. 2f, it can be concluded that the dsDNA stands perpendicular to graphene.

### Example 2

Because dsDNA bending has been shown to be crucial for different biological functions, the following example exploits the perpendicular orientation of dsDNA on graphene to develop a platform to measure bending angles of dsDNA. Inspired by the well-defined orientation of dsDNA achieved with GETvNA, a pipeline to measure bends on dsDNA has been developed, which are crucial for many biological functions, such as gene expression (Pérez-Martin J, et al., Microbiological Reviews, 1994, 58: 268-290; Pérez-Martin J and de Lorenzo V, Annual Review of Microbiology, 1997, 51:593-628; Kim J L, et al., Nature, 1993, 365:520-527), and DNA repair regulations (Garcin E D, et al., Nature Structural & Molecular Biology, 2008, 15:515-522; Sharma M, et al., Journal of Physical Chemistry B, 2013, 117:6194-6205; Janićijević A, et al., DNA Repair (Amst), 2003, 2:325-336; Hosfield D J, et al., Cell, 1999, 98:397-408; Missura M, et al., EMBO Journal, 2001, 20:3554-3564), or DNA packaging in viral capsids (Petrov A S and Harvey S C, Biophysical Journal, 2008, 95:497-502; Tzlil S, et al., Biophysical Journal, 2003, 84:1616-1627), and nucleosomes (Drew H R and Travers A A, Journal of Molecular Biology, 1985, 186:773-790; Widom J, Quarterly Reviews of Biophysics, 2001, 34: 269-324). While different techniques have been used to study DNA bending (Vermeulen A, et al., Journal of the American Chemical Society, 2000, 122:9638-9647; Demurtas D, et al., Nucleic Acids Research, 2009, 37: 2882-2893; Reinhard B M, et al., Proceedings of the National Academy of Sciences, 2007, 104: 2667-2672; Rees W A, et al., Science, 1993, 260:1646-1649; Harrington R E, Electrophoresis, 1993, 14:732-746; Wiggins P A, et al., Nature Nanotechnology, 2006, 1:137-141), FRET has been the method of choice to observe dynamic changes of DNA bends at the single-molecule level. However, when studying a kinked dsDNA using FRET, additional rotations or translations between the segments located at both sides of the kink can affect the donor-acceptor distance. Therefore, the direct calculation of the angle from the FRET efficiency is hindered, and comparisons with simulations or complementary experiments are needed to get accurate angles.

Here, it will be demonstrated that GETvNA overcomes these limitations and allows monitoring bending angles in real-time, at the single-molecule level, and in a model-free fashion.

First, the method was tested by measuring the bending angle of dsDNA molecules containing kinks originating from the presence of A bulges, namely unpaired adenines (3, 5 or 7 adenines). To form the hybrid molecule used in these experiments, we combined a 66 nucleotides long ssDNA (66mer) (SEQ ID NO: 4) functionalized at the 5′ end with Cy3B (purchased at Eurofins) with longer ssDNA molecules containing 139 (SEQ ID NO: 5), 141 (SEQ ID NO: 6), and 143 (SEQ ID NO: 7) nucleotides (purchased at Integrated DNA Technologies) (see Table 1). In the longer ssDNA molecules, there was a segment of 70 nucleotides that had no complementarity to the 66mer and gave rise to the ssDNA overhang to attach the hybrid molecule to graphene. The rest of the ssDNA contained 69, 71 and 73 nucleotides (for the 139, 141 and 143mer, respectively), and in all cases 66 of them were complementary to the shorter ssDNA labeled with Cy3B. The extra nucleotides that were not complementary to the shorter ssDNA were 3, 5 and 7 consecutive adenines located at 30 nucleotides from the 5′ end, and were the bases that gave origin to the bulges. In FIG. 3a, a sketch of the experimental design is shown, where one dsDNA segment stands perpendicular on graphene and, after the bulge, a second dsDNA segment extends following a given kink angle, θ. This angle can be calculated from the height of the dye through Equation 3:

$$θ = arcsin ( \frac{z - {z}_{kink}}{L - {z}_{kink}} ) \qquad ( 3 )$$

In this system, zₖᵢₙₖ can be estimated as 11.9 nm and L as 20.82 nm using the Worm-like chain model. In FIG. 3b, the dependence of the height on the bending angle is shown, which was analytically obtained from Equation 3. From this plot, it can be concluded that the method is more sensitive in the 30-120° range than below 30°.

Bulges with 3, 5 and 7 adenines (hereafter called A3, A5, and A7) were tested. These kinds of kinks have been measured by experimental techniques, such as gel electrophoresis, NMR, FRET, and single-molecule FRET. In the cases where FRET was used, a broad range of angles were reported, due to the multiple possible conformations that can give rise to the same FRET efficiency (Woźniak A K, et al., Proceedings of the National Academy of Sciences, 2008, 105:18337-18342; Gohlke C, et al., Proceedings of the National Academy of Sciences, 1994, 91:11660-11664). In contrast, because of the planar geometry of graphene, the rotations and translations of the dsDNA segments that are detrimental for FRET-based angle measurements do not have a strong impact on the results obtained using GET. In simulations, it was demonstrated that the inaccuracy of GETvNA is typically below 10°, while in FRET this value can increase up to 60° due to the higher conformational ambiguity.

In the present Example, single-molecule lifetime measurements were carried out under the same conditions and with identical equipment as in Example 1. In FIG. 3d, the height histograms obtained for the three samples are depicted. Compared to the distribution obtained in the absence of bulges (FIG. 1d, 66 bp), the axial positions of the fluorophores were shifted closer to graphene. This behaviour is compatible with the schemes from FIG. 3c, where the dsDNA is progressively bent to higher angles when moving from A3 to A7, as it was also reported in previous studies (Woźniak A K, et al., Proceedings of the National Academy of Sciences, 2008, 105:18337-18342; Gohlke C, et al., Proceedings of the National Academy of Sciences, 1994, 91:11660-11664; Gu H, et al., Journal of the American Chemical Society, 2010, 132:4352-4357; Lilley D M, Proceedings of the National Academy of Sciences, 1995, 92:7140-7142).

The distributions of bending angles are plotted in FIG. 3e, where two subpopulations for both A3 and A5 are visible. Therefore, two-peak Gaussian fits were performed and yielded mean angles of 56.8° (σ_{A3,main}=) 17.5° and 69.9° (σ_{A5,main}=) 16.2° for the main populations of A3 and A5, respectively. These values are in good agreement with the ones reported by Woźniak, et al., which are 56° and 73° (Woźniak A K, et al., Proceedings of the National Academy of Sciences, 2008, 105:18337-18342). On the other hand, the A7 population and the least representative A3 and A5 populations were centered at similar angular values: 94.8°, 94.5°, and 94.2° for A3, A5, and A7, respectively. In all three cases, the width of the distributions was narrower than those of the main populations of A3 and A5 (σ_{3A,minor}=6.0°, σ_{5A,minor}=4.3°, and σ_{7A}=) 9.0°. Since the precision of the angular measurements is not expected to be higher for 94° than for 56°-70°, the fact that the main populations of A3 and A5 showed broader distributions can be explained by a true molecular heterogeneity, given by an energy landscape that allows different stable configurations to coexist at room temperature. Regarding the mean value obtained for A7, it coincides with the results reported in the literature (85°, 90° and)>85-105° (Gohlke C, et al., Proceedings of the National Academy of Sciences, 1994, 91:11660-11664; Gu H, et al., Journal of the American Chemical Society, 2010, 132:4352-4357; Lilley D M, Proceedings of the National Academy of Sciences, 1995, 92:7140-7142).

The detection of subpopulations for A3 and A5 is a noteworthy achievement. This would not have been possible if it would have been necessary to extract average energy transfer efficiency values to fit a geometrical model or compare to simulations. The ability to analyze each individual molecule and to determine its angle allows detecting subpopulations and comparing distribution widths, which in turn provides valuable insights into the molecular geometry of kinked dsDNA.

Second, it was studied how dsDNA with an abasic (AP) site was bent due to the binding of a mutant Escherichia coli endonuclease IV (Endo IV), and values that agree with the ones reported from X-ray crystallography for the wild-type enzyme were obtained. To obtain the hybrid molecule, we mixed a 136 nucleotides long ssDNA (purchased at Integrated DNA Technologies) (SEQ ID NO: 1) with a 66 nucleotides long ssDNA (SEQ ID NO: 8), functionalized with Cy3B at the 5′ end and containing a dSpacer (Spd, which serves as AP site) modification at the position 22 counting from the 5′ end (purchased at Eurofins) (see Table 1). The hybrid molecule, therefore, contains a 70 nucleotides ssDNA overhang to attach to graphene and a dsDNA segment of 66 base pairs. The endonuclease IV gene from Escherichia coli was PCR amplified from chromosomal DNA and cloned between the Ncol Xhol restriction sites of the expression plasmid pET24-d (Novagen, Merck Millipore, Germany). The E261Q endonuclease IV activity mutant was created by overlapping PCR and cloned in the pET24-d vector using the same restriction sites. All enzymes for cloning were purchased from New England Biolabs (MA, USA) and the E. coli XL1blue strain was used for cloning. The E261Q endonuclease IV mutant was produced in the E. coli expression strain BL21 Star (DE3). The cells were grown in LB supplemented with 30 μg/ml of kanamycin at 37° C. When the cell culture reached an OD₆₀₀ of 1, the expression was induced with 1 mM IPTG for 4 hours at 25° C. The protein was purified by metal affinity chromatography. After the affinity purification, the sample was further purified by cationic exchange chromatography using HiScreen SP HP columns (Cytiva Europe GmbH, Germany). The cationic exchange chromatography was performed in 20 mM Tris, 100 mM NaCl, 0.2 mM EDTA pH 7.4 and the protein was eluted from the column using a linear gradient to a 20 mM Tris, 1 M NaCl, 0.2 mM EDTA buffer pH 7.4. The sample was stored at −20° C. in 50% glycerol 10 mM Tris, 50 mM NaCl, 0.1 mM EDTA pH 7.4.

In the publications where the crystallographic structure of the Endo IV bound to dsDNA was reported, the authors measured a bending angle of around 90° (Garcin E D, et al., Nature Structural & Molecular Biology. 2008, 15:515-522; Hosfield D J, et al., Cell, 1999, 98:397-408).

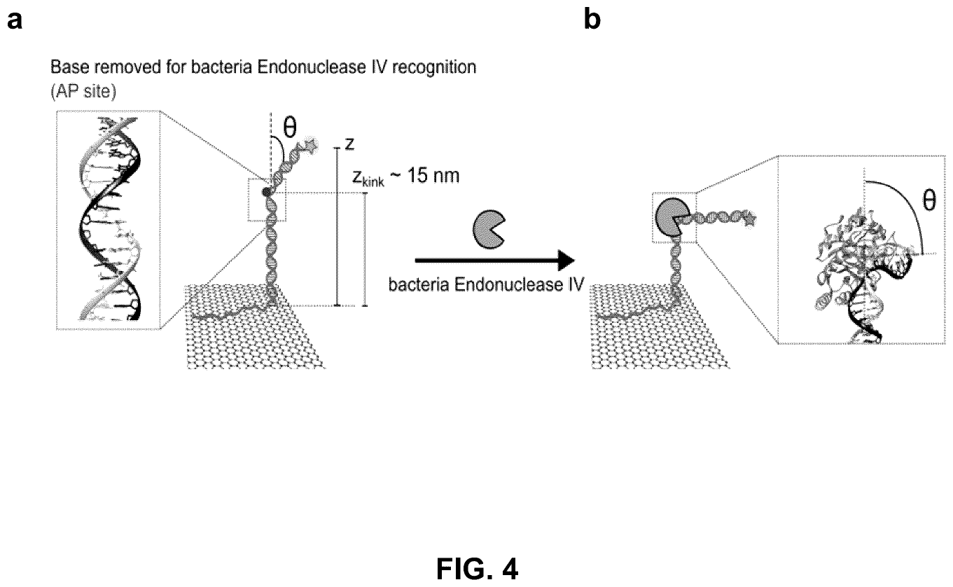

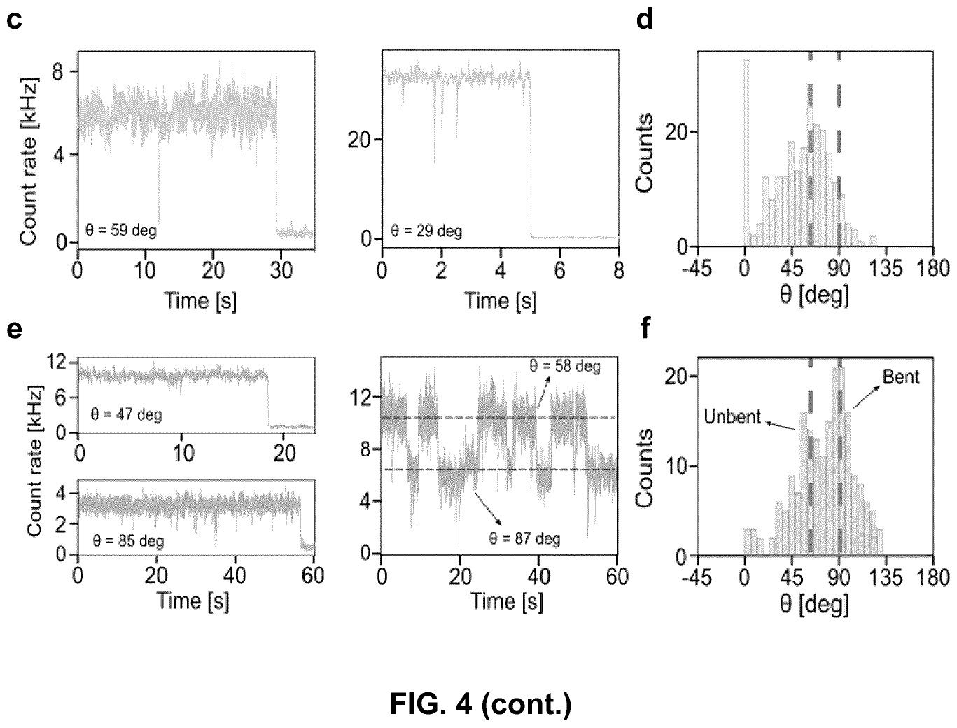

In FIGS. 4a and 4b, the experimental design is shown that was used to study the bending of Endo IV using GETvNA. In FIGS. 4c and 4e, representative single-molecule fluorescence intensity time traces in the absence and presence of Endo IV are presented, respectively. In the traces from FIG. 4c (absence of enzyme), it can be observed that before adding Endo IV, the dsDNA is already bent to some extent (the two example traces show bending angles of 59° and) 29° graphene. This observation can be explained by the fact that the AP site already breaks the TT-stacking of the dsDNA, which allows for an increased bendability of the DNA, as previously reported in NMR studies (Chen J, et al., Biochemistry, 2007, 46:3096-3107).

After adding the enzyme, bending angles of up to 87° can be obtained, as shown in the exemplary traces from FIG. 4e. In FIGS. 4d and 4f, the histograms of the bending angle obtained for 250 single molecules in the absence and presence of the enzyme are presented, respectively. The median angle of the dsDNA with AP site and with no Endo IV added to the system is 55°. On the other hand, after adding Endo IV, the peak at 55° is still observed, but a new maximum centered at about 90° can be detected, in agreement with the expected bending angle of a DNA bound to Endo IV. Therefore, it has been confirmed that GETvNA allows measuring bending angles of dsDNA due to enzymatic activity. Moreover, it is possible to detect several bending events of the same dsDNA and study the dynamics of this process, as shown in the right panel of FIG. 4e.

### Example 3

The following example investigates diffusion of the O⁶-alkylguanine DNA alkyltransferase (AGT) along the dsDNA segment as second application for GETvNA.

AGT is a protein involved in repairing alkylguanine lesions that can be highly mutagenic and cytotoxic (Kono S, et al., Proceedings of the National Academy of Sciences, 2022, 119: e2116218119). Understanding its diffusion once it is bound to DNA can shed light on the lesion search and repair mechanism of AGT. These kinds of studies are usually performed using single-molecule FRET or single-molecule tracking experiments in camera-based optical setups. Compared with FRET, GETvNA spans a distance range that is 4 to 5-fold larger. On the other hand, when compared with camera-based single-molecule tracking measurements, GETvNA is 10 to 20 times more photon efficient, meaning that with the same photon counts, GETvNA will be 10 to 20 times more precise to localize the position of the protein on the DNA. Applying GETvNA, it was possible to track individual AGT molecules as well as AGT clusters, with millisecond time resolution and single-base pair spatial resolution. This performance excels the ones achieved with other tracking techniques, including the ones obtained with MINFLUX, a recently developed method that stands out among the state-of-the-art tracking techniques (Zähringer J. et al., Light: Science & Applications, 2023, 12:70; Deguchi T. et al., Science, 2023, 379:1010-1015; Wolff J O, et al., Science, 2023, 379:1004-1010).

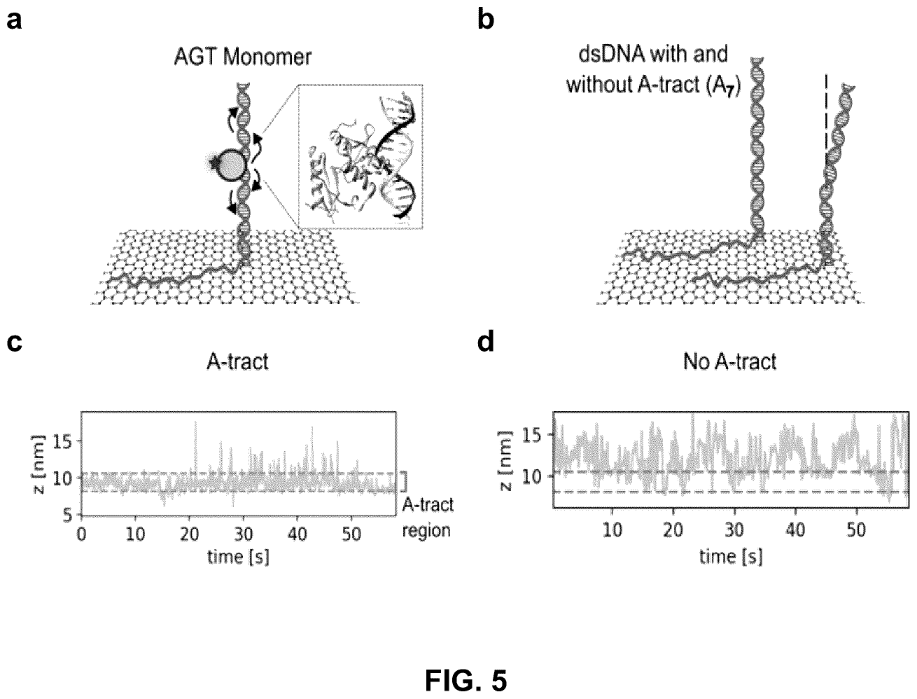

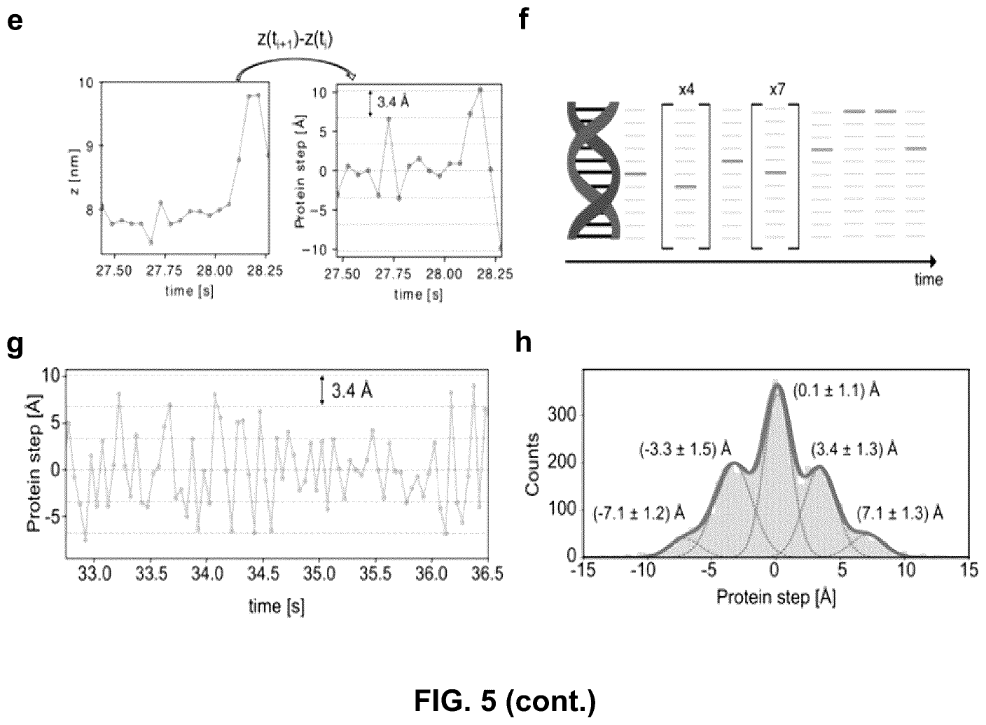

In the present Example, single-molecule lifetime measurements were carried out under the same conditions and with identical equipment as in Example 1. The experimental design to study the diffusion of AGT on a vertically oriented dsDNA is shown in FIG. 5a, where it can be seen that AGT is fluorescently labeled (in this case, with a dye molecule ATTO647N. The hybrid molecules were formed by combining a 136 nucleotides long ssDNA (Integrated DNA Technologies) (SEQ ID NO: 1) with a 66 nucleotides long ssDNA (Eurofins) containing or lacking an A-tract (SEQ ID NO: 9; SEQ ID NO: 10) (see Table 1). As a result, the hybrid molecules had a 70 nucleotides long overhang to attach to graphene and a dsDNA segment of 66 base pairs. A C145S mutant variant of AGT was labeled through one available cysteine site with a maleimide version of ATTO647N. AGT was added at room temperature, at a 400 nM concentration in a buffer containing 10 mM Tris pH 7.9, 50 mM NaCl, and 1 mM dithiothreitol (DTT). After 2 minutes of incubation, the sample was washed 10 times with the same incubation buffer, and then single-molecule measurements were performed in the same way as in the previous examples. As depicted in FIG. 5b, the diffusion on dsDNA containing and lacking an A-tract was studied to investigate the influence of having a ˜20° bent on the double-helix. In FIGS. 5c and 5d, typical time traces on DNAs with and without A-tract are presented, where it can be seen that the protein moves in both directions and that there are two diffusive regimes: a slow one and a fast one. Additionally, in the case where there is an A-tract on the DNA, an increased affinity to the part of the DNA where the A-tract is located can be seen, which represents a new finding that was not reported by others so far.

Finally, the protein steps of slow trajectories were studied, namely the difference in height between each time bin (50 ms) and the following one. In FIG. 5e, a short segment of a time trace (<1 s) is shown, and it was depicted how to obtain the protein steps directly from the time trace. As can be seen in the protein steps plot, it is possible to distinguish the movements that matched the distance corresponding to multiples of single base pairs. In the sketch from FIG. 5f, the up- and downwards movement performed by the individual AGT enzymes is presented within the time window shown in FIG. 5e. The protein step plot from FIG. 5g shows a larger time window, where the single-base pair resolution can still be observed. Finally, multiple slow trajectories (260 seconds total time) were analyzed, and a histogram of protein steps was made, which is shown in FIG. 5h. Therein, a 5-peak Gaussian fit of the obtained distribution is presented, where steps of −7.1, −3.3, 0.1, 3.4, and 7.1 Ångström can be observed, which perfectly match with steps of −2, −1, 0, 1 and 2 base pairs. This is a true novelty in fluorescence microscopy to track entities with single-digit Ångström spatial resolution at the available photon count, and without highly specialized and costly equipment.

### Example 4

The following example investigates the orientation of RNA on graphene. To determine the orientation of RNA on graphene, it was measured how efficiently the emitted energy of single fluorophores located at the end of the dsRNA segment is transferred to graphene (FIG. 6). To investigate the actual orientation of the double helix, a fluorescent dye (ATTO532) was attached to one of its end bases. Single-molecule fluorescence lifetime measurements were then performed. The graphene substrate was produced as described in Example 1. To prepare the hybrid RNA molecule, custom ssRNA sequences were chosen. Single-stranded RNA molecules were ordered from IDT Technologies. Those strands were hybridized with the same protocol as for the dsDNA as described above, but with a buffer containing 1xTAE and 50 mM NaCl. For a buffer dependency test, another buffer with 500 mM NaCl was used. dsRNA molecules of three different lengths of 40, 60 and 80 base pairs (bp) were studied. To obtain these samples, different ssRNA molecules were combined to hybrid molecules. In all cases, the same 100 nucleotides long ssRNA was used (SEQ ID NO: 11), which was hybridized with three different ssRNA with lengths of 40 nt (SEQ ID NO: 12), 60 nt (SEQ ID NO: 13) and 80 nt (SEQ ID NO: 14) functionalized at the 5′ end with ATTO542 (see Table 1). As a result, the obtained hybrid molecules contained dsRNA segments of 40, 60 and 80 base pairs and ssRNA overhangs of at least 19 nucleotides. The annealing of the RNA and the immobilization on the graphene surface were performed under RNase-free conditions to ensure the integrity of the RNA samples. A 1× TEA 50 mM NaCl buffer was used for annealing and storage of the sample and a 1× TAE 50 mM NaCl/2 mM Trolox/12 mM PCA/1× PCD buffer was used for fluorescence measurement. FLIM (Fluorescence Lifetime Imaging Microscopy) images were obtained under the same conditions and with identical equipment as in Example 1. In FIG. 6a, an exemplary FLIM image acquired for the 80mer sample is shown, where homogeneous lifetime values can be observed.

The resulting plots are displayed in FIGS. 6a to 6g. The results were obtained from time-intensity traces acquired for each individual molecule and deconvoluted with the instrument response function. The fluorescence lifetime of the signals was used to calculate the distance between the graphene surface and the fluorescent dye, also referred to as height. Defined fluorescence lifetimes were obtained with RNA. It indicates that RNA stands perpendicular to graphene. Small deviations to expected heights are related to the fact that the contour length of RNA is less defined. Additionally, RNA has a higher tendency to adopt alternative conformations explaining more heterogeneity. The fact that defined populations are obtained with heights around the expected values for straight RNA can only be explained by the fact that RNA behaves fundamentally similar to DNA with respect to its interaction with graphene and that the lowest double-stranded basepair is firmly attached to the graphene orienting the whole molecule. For these reasons, the method is promising for detecting RNAs as well as for obtaining structural information with respect to their secondary and tertiary structure.

### Example 5

In the following Example, it is demonstrated that GETvNA allows detecting single-nucleotide variations on dsDNA through the bending of the respective strands.

The graphene substrate has been produced as described in Example 1. To form the hybrid molecule, we combined a 76 nucleotides long ssDNA (SEQ ID NO: 15) functionalized at the 3′ end with ATTO542 with a 51 ssDNA molecule containing zero mismatches (SEQ ID NO: 16), one mismatch (SEQ ID NO: 17), or two adjacent G-A and A-G mismatches (SEQ ID NO: 18). The ssDNA-dsDNA hybrid molecule for the detection of DNA mismatches was annealed and immobilized on the graphene surface in the same way as described in the preceding Examples.

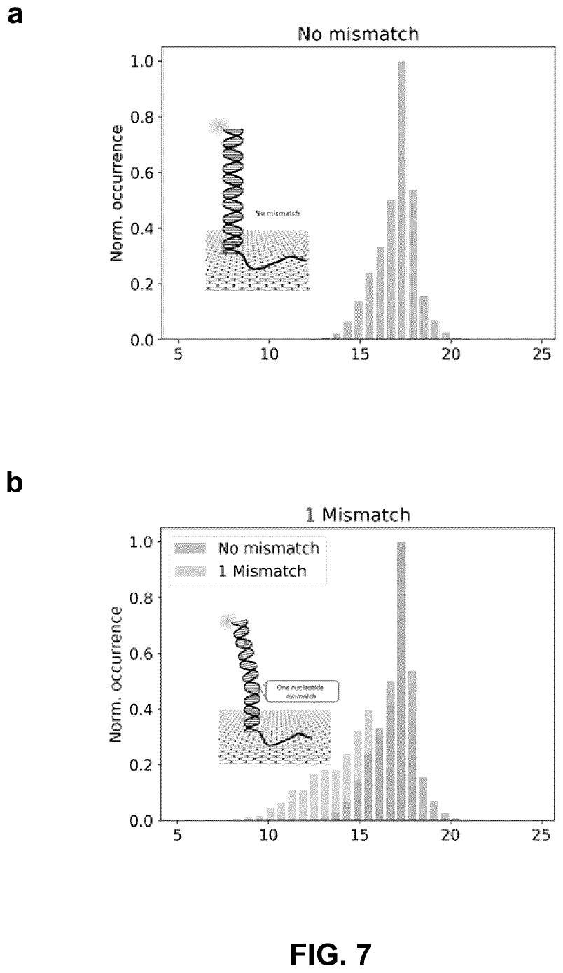

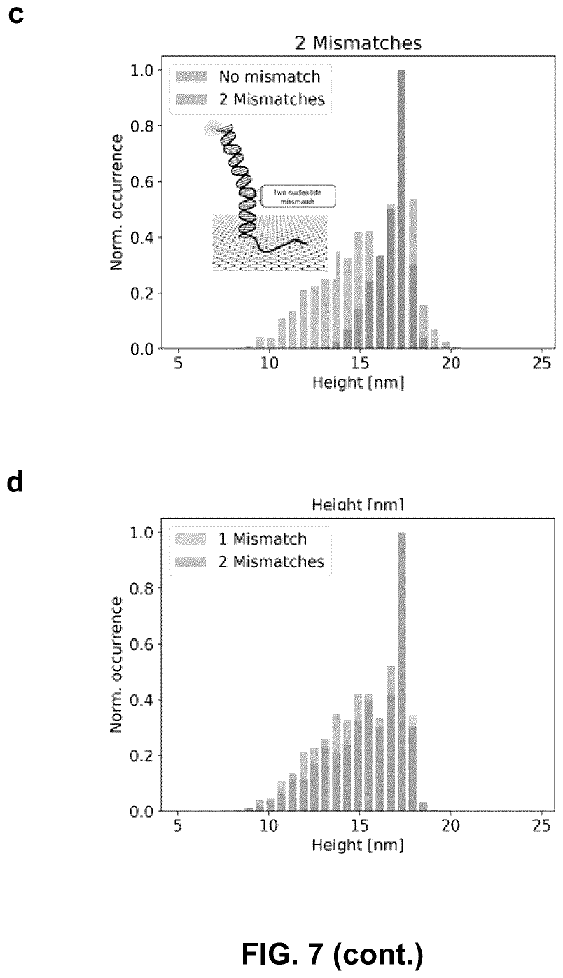

The results shown in FIG. 7 were obtained from FLIM scans and deconvoluted with the instrument response function as described in the preceding Examples. The fluorescence lifetime of the signals was used to calculate the distance between the graphene surface and the fluorescent dye, also referred to as height as described earlier. The different distributions obtained indicate that GETvNA can be used to specifically detect nucleotide mismatches in nucleic acid.

TABLE 1
Summary of the nucleic acid sequences used in the Examples.
“Spd” refers to a dSpacer, which served as abasic (AP) site
(the symbol “T” in the corresponding sequence listing will be
construed as thymine in DNA and uracil in RNA).

| | Hybrid | Olignonucleotide 1 | Olignonucleotide 2 |
|---|---|---|---|
| Example | Molecule | (ssDNA) (5′ to 3′) | (ssDNA) (5′ to 3′) |
| Example 1 | 36mer | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC |
| | | CTTAAGAACTGGCTACGACG | (SEQ ID NO: 2) |
| | | GACTGATCGGATCGAATAAC | |
| | | GAGTACACGGTGGTTA | |
| | | (SEQ ID NO: 1) | |
| | 51mer | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC CGT CGT |
| | | CTTAAGAACTGGCTACGACG | AGC CAG TTC |
| | | GACTGATCGGATCGAATAAC | (SEQ ID NO: 3) |
| | | GAGTACACGGTGGTTA | |
| | | (SEQ ID NO: 1) | |
| | 66mer | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC CGT CGT |
| | | CTTAAGAACTGGCTACGACG | AGC CAG TTC TTA |
| | | GACTGATCGGATCGAATAAC | AGA CTG TAG ATT |
| | | GAGTACACGGTGGTTA | (SEQ ID NO: 4) |
| | | (SEQ ID NO: 1) | |
| Example 2 | Bulge A3 | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC CGT CGT |
| | | CTTAAGAACTGGCTACGACG | AGC CAG TTC TTA |
| | | GACTGATAAACGGATCGAAT | AGA CTG TAG ATT |
| | | AACGAGTACACGGTGGTTA | (SEQ ID NO: 4) |
| | | (SEQ ID NO: 5) | |
| | Bulge A5 | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC CGT CGT |
| | | CTTAAGAACTGGCTACGACG | AGC CAG TTC TTA |
| | | GACTGATAAAAACGGATCGA | AGA CTG TAG ATT |
| | | ATAACGAGTACACGGTGGTT | (SEQ ID NO: 4) |
| | | A | |
| | | (SEQ ID NO: 6) | |
| | Bulge A7 | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT TCG ATC CGA |
| | | GGGTTGGATAAATCTACAGT | TCA GTC CGT CGT |
| | | CTTAAGAACTGGCTACGACG | AGC CAG TTC TTA |
| | | GACTGATAAAAAAACGGATC | AGA CTG TAG ATT |
| | | GAATAACGAGTACACGGTGG | (SEQ ID NO: 4) |
| | | TTA | |
| | | (SEQ ID NO: 7) | |
| | AP site | ACGGTCGTCAAATCTGTCGT | [Cy3B] TAA CCA |
| | | GGTAGGATCTGTCGTGGTAG | CCG TGT ACT CGT |
| | | GATCTGTCGTGGTAACTTTG | TAT [Spd]CG ATC |
| | | GGGTTGGATAAATCTACAGT | CGA TCA GTC CGT |
| | | CTTAAGAACTGGCTACGACG | CGT AGC CAG TTC |
| | | GACTGATCGGATCGAATAAC | TTA AGA CTG TAG |
| | | GAGTACACGGTGGTTA | ATT |
| | | (SEQ ID NO: 1) | (SEQ ID NO: 8) |
| Example 3 | AGT | ACGGTCGTCAAATCTGTCGT | TAA CCA CCG TGT |
| | NoAtract | GGTAGGATCTGTCGTGGTAG | ACT CGT TAT TCG |
| | | GATCTGTCGTGGTAACTTTG | ATC CGA TCA GTC |
| | | GGGTTGGATAAATCTACAGT | CGT CGT AGC CAG |
| | | CTTAAGAACTGGCTACGACG | TTC TTA AGA CTG |
| | | GACTGATCGGATCGAATAAC | TAG ATT |
| | | GAGTACACGGTGGTTA | (SEQ ID NO: 4) |
| | | (SEQ ID NO: 1) | |
| | AGT | ACGGTCGTCAAATCTGTCGT | TAA CCA CCG TGT |
| | Atract | GGTAGGATCTGTCGTGGTAG | ACT CGT TAT TCG |
| | | GATCTGTCGTGGTAACTTTG | ATC CGA TCA GTC |
| | | GGGTTGGATAAATCTACAGT | AAA AAA AGC CAG |
| | | CTTAAGAACTGGCTTTTTTT | TTC TTA AGA CTG |
| | | GACTGATCGGATCGAATAAC | TAG ATT |
| | | GAGTACACGGTGGTTA | (SEQ ID NO: 10) |
| | | (SEQ ID NO: 9) | |
| Example 4 | 40mer | UCG CCA ACA GCU CCG | [ATTO532]CGC AUG |
| | | CGC GAG CAC GAG CGA | UUC GGC GCA GCU |
| | | AGU CAA CUG CUG GAC | AGU GCA AAC CCU |
| | | ACC UGA CGA CCU GAU | GCA AUC ACG G |
| | | CCG UGA UUG CAG GGU | (SEQ ID NO: 12) |
| | | UUG CAC UAG CUG CGC | |
| | | CGA ACA UGC G | |
| | | (SEQ ID NO: 11) | |
| | 60mer | UCG CCA ACA GCU CCG | [ATTO532]CGC AUG |
| | | CGC GAG CAC GAG CGA | UUC GGC GCA GCU |
| | | AGU CAA CUG CUG GAC | AGU GCA AAC CCU |
| | | ACC UGA CGA CCU GAU | GCA AUC ACG GAU |
| | | CCG UGA UUG CAG GGU | CAG GUC GUC AGG |
| | | UUG CAC UAG CUG CGC | UGU CCA |
| | | CGA ACA UGC G | (SEQ ID NO: 13) |
| | | (SEQ ID NO: 11) | |
| | 80mer | UCG CCA ACA GCU CCG | [ATTO532]CGC AUG |
| | | CGC GAG CAC GAG CGA | UUC GGC GCA GCU |
| | | AGU CAA CUG CUG GAC | AGU GCA AAC CCU |
| | | ACC UGA CGA CCU GAU | GCA AUC ACG GAU |
| | | CCG UGA UUG CAG GGU | CAG GUC GUC AGG |
| | | UUG CAC UAG CUG CGC | UGU CCA GCA GUU |
| | | CGA ACA UGC G | GAC UUC GCU CGU |
| | | (SEQ ID NO: 11) | GC |
| | | | (SEQ ID NO: 14) |
| Example 5 | No | GGG GTT GGA TAA ATC TAC | AAC CAC CGT GTA |
| | mismatch | AGT CTT ACG CGC TGG CTA | CTC GTT ATT CGA |
| | | CGA CGG ACT GAT CGG | TCC GAT CAG TCC |
| | | ATC GAA TAA CGA GTA CAC | GTC GTA GCC AGC |
| | | GGT GGT T (ATTO542) | GCG |
| | | (SEQ ID NO: 15) | (SEQ ID NO: 16) |
| | One | GGG GTT GGA TAA ATC TAC | AAC CAC CGT GTA |
| | mismatch | AGT CTT ACG CGC TGG CTA | CTC GTT ATT CGA |
| | | CGA CGG ACT GAT CGG | TCC GAT CAG TCC |
| | | ATC GAA TAA CGA GTA CAC | GTC GAA GCC AGC |
| | | GGT GGT T (ATTO542) | GCG |
| | | (SEQ ID NO: 15) | (SEQ ID NO: 17) |
| | Two | GGG GTT GGA TAA ATC TAC | AAC CAC CGT GTA |
| | mismatches | AGT CTT ACG CGC TGG CTA | CTC GTT ATT CGA |
| | | CGA CGG ACT GAT CGG | TCC GAT CAG TCC |
| | | ATC GAA TAA CGA GTA CAC | GTC AGA GCC AGC |
| | | GGT GGT T (ATTO542) | GCG |
| | | (SEQ ID NO: 15) | (SEQ ID NO: 18) |

1. A construct comprising a graphene substrate and a hybrid molecule comprising a single-stranded nucleic acid segment and a double-stranded nucleic acid segment, wherein the hybrid molecule is immobilized to the graphene substrate by the single stranded nucleic acid segment, and wherein the double-stranded nucleic acid segment comprises a linear segment comprising at least one base pair in perpendicular orientation to the graphene substrate at the junction where the double-stranded nucleic acid segment and the single-stranded nucleic acid segment join.

2. The construct of claim 1, wherein the nucleic acid is selected from the group consisting of DNA and RNA.

3. The construct of claim 1, wherein the single-stranded nucleic acid segment is a single-stranded DNA segment, and the double-stranded nucleic acid segment is a double-stranded DNA segment.

4. The construct of claim 1, wherein the first base pair of the double-stranded nucleic acid segment of the hybrid molecule at the junction is immobilized to the graphene substrate.

5. The construct of claim 1, wherein the graphene substrate comprises at least a monolayer of graphene on a matrix.

6. The construct of claim 1, wherein the double-stranded nucleic acid segment comprises at least one bend and a further linear segment extending from the bend having a non-perpendicular orientation to the graphene substrate.

7. The construct of claim 6, wherein the bend originates from the presence of unpaired mismatches, abasic sites, A-tracts, nicks, or binding of different proteins that interact with nucleic acid.

8. The construct of claim 1, wherein the double-stranded nucleic acid segment comprises at least one photoluminescent particle.

9. The construct of claim 8, wherein the at least one photoluminescent particle is attached directly to the double-strand nucleic acid segment or is attached indirectly to the double-strand nucleic acid segment via a protein bound to the double-strand DNA segment.

10. The construct of claim 8, wherein the at least one photoluminescent particle is a fluorophore or a quantum dot.

11. The construct of claim 1, wherein the linear segment comprised in the double-stranded nucleic acid segment is at least 15 base pairs long.

12. The construct of claim 1, wherein the single-stranded nucleic acid segment is between 5 and 100 nucleotides in length.

13. The construct of claim 1, wherein the distance of the photoluminescent particle to graphene is at least 5 nm.

14. A method for producing the construct of claim 1, wherein the method comprises the step of (i) or (ii):
(i) mixing a graphene substrate and a hybrid molecule comprising a single-stranded nucleic acid segment and a double-stranded nucleic acid segment to allow immobilization of the single-stranded nucleic acid segment to the graphene substrate; or
(ii) adding a first single-stranded nucleic acid to a graphene substrate to allow attachment of the first single-stranded nucleic acid to the graphene substrate, and adding a second single-stranded nucleic acid complementary to the first single-stranded nucleic acid to form a hybrid molecule comprising a single-stranded nucleic acid segment and a double-stranded nucleic acid segment.

15. A method for measuring quenching efficiency by means of measuring the fluorescence lifetime and/or fluorescence intensity in the construct of claim 8.

16. The method of claim 15, wherein fluorescence lifetime and/or fluorescence intensity is measured by using Fluorescence Lifetime Imaging Microscopy.

17. A microscopy system for measuring fluorescence lifetime in the construct of claim 8.

18. The microscopy system of claim 17, comprising:
an excitation path, an objective, a detection path, a detector, and a Time Correlated Single Photon Counting unit;
wherein the excitation path comprises a laser and is configured to produce a pulsed laser beam and direct the pulsed laser beam into the objective using one or more beam redirecting elements, the pulsed laser beam having a pulse width below 750 ps, preferably below 500 ps, more preferably below 200 ps;
wherein the objective is configured to transmit the pulsed laser beam into a sample comprising the construct and to collect light from the sample, particularly light emitted from the construct;
wherein the detection path is configured to direct light from the objective, particularly the light from the sample, to the detector using one or more beam redirecting elements;
wherein the detector is configured to detect the light received from the detection path, particularly the light from the sample, that is directed onto, e.g. focused on, the detector using a lens; and
wherein the microscopy system is configured to measure an arrival time of individual photons at the detector after a laser pulse, using the Time Correlated Single Photon Counting unit.

19. The microscopy system of claim 18, wherein the average power of the pulsed laser beam is 10 μW or less at the position of the objective.

20. The microscopy system of claim 18, wherein the detection path is configured to focus the light from the objective into a point-like detector.
